(19) United States
(12) Patent Application Publication
(10) Pub. No.: US 2008/0039492 A1
(43) Pub. Date: Feb. 14, 2008

(54) Quinine products, method of manufacture, and method of use

(76) Inventors: Richard Howard Roberts, Lakewood, NJ (US); Jie Du, Lansdale, PA (US); Matthew William Davis, Erwinna, PA (US); Kurt R. Nielsen, Chadds Ford, PA (US)

Correspondence Address: CANTOR COLBURN, LLP, 55 GRIFFIN ROAD SOUTH, BLOOMFIELD, CT, 06002, US

(21) Appl. No.: 11/492,299

(22) Filed: Jul. 25, 2006

(51) Int. Cl. A61K 31/4745; A61B 5/00

(52) U.S. Cl. 514/305; 600/300

(57) ABSTRACT

Disclosed herein is a method of using quinine. In one embodiment, the method comprises obtaining quinine from a container associated with published material providing information that quinine affects the activity of a cytochrome p450 isozyme. In another embodiment, the method comprises informing a user that quinine affects the activity of a cytochrome p450 isozyme. Also included are articles of manufacture comprising a container containing a dosage form of quinine, wherein the container is associated with published material informing that quinine affects activity of a cytochrome p450 isozyme. Also disclosed are a method of treatment and a method of manufacturing a quinine product.

### BACKGROUND

This application relates to quinine products for therapeutic purposes, and in particular to improved methods of use of quinine sulfate.

Malaria is a parasitic disease caused by the Plasmodium species P. falciparum, P. vivax, P. ovale, and P. malariae. The malaria parasite causes intermittent fevers and chills. It affects multiple organs and systems, including red blood cells, the kidneys, liver, spleen, and brain. It is estimated by the World Health Organization (WHO) that up to 500 million persons per year are infected with malaria, with 200 to 300 million people suffering from malaria at any given time. Up to 3 million will die each year. If P. falciparum infection goes untreated or is not treated appropriately, general observations indicate that mortality is high, killing up to 25% of non-immune adults within 2 weeks of a primary attack [Taylor T E, Strickland G T. Malaria. In: Strickland G T, ed. Hunter's Tropical Medicine and Emerging Infectious Diseases. 8th ed. Philadelphia, Pa.: W.B. Saunders Company; 2000.] A significant number of these cases are found in Central America, South America, Asia, and Africa. Known antimalarial agents include 9-aminoacridines (e.g. mepacrine), 4-aminoquinolines (e.g. amodiaquine, chloroquine, hydroxychloroquine), 8-aminoquinolines (e.g. primaquine, quinocide), biguanides with an inhibiting effect on dihydrofolic acid reductase (e.g. chlorproguanil, cycloguanil, proguanil), diaminopyrimidines (e.g. pyrimethamine), quinine salts, sulphones such as dapsone, sulphonamides, sulphanilamides, and antibiotics such as tetracycline.

Quinine (cinchonan-9-ol, 6′-methoxy-, (8a,9R)—) is an antiprotozoal and an antimyotonic, and is known for the treatment of malaria caused by Plasmodium species, the treatment and prophylaxis of nocturnal recumbency leg muscle cramps, and the treatment of babesiosis caused by Babesia microti. Quinine is structurally similar to quinidine, which is also an antiprotozoal, but can function as an antiarrhythmic. Quinidine has been associated with the prolongation of the QT interval in a dose-related fashion. Excessive QT prolongation has been associated with an increased risk of ventricular arrhythmia. Although quinine is a diastereomer of quinidine, it does not cause QT prolongation to the same degree although it has been suggested that patients with a history of cardiac arrhythmias and/or QT prolongation should carefully consider taking quinine as they may be at risk for arrhythmias.

Quinine sulfate is currently supplied in the United States as capsules for oral administration containing 324 milligrams (mg) of quinine sulfate USP, equivalent to 269 mg of the free base. For treatment of uncomplicated P. falciparum malaria in adults, the dosage of quinine sulfate is 648 mg (two capsules) every 8 hours for 7 days.

One of the most important groups of Phase I metabolic enzymes are the cytochrome p450 monooxygenase system enzymes. The cytochrome p450 enzymes are a highly diverse superfamily of enzymes. NADPH is required as a coenzyme and oxygen is used as a substrate. Each enzyme is termed an isoform or isozyme since each derives from a different gene.

Many members of the cytochrome p450 family are known to metabolize active agents in humans. Active agent interactions associated with metabolism by cytochrome p450 isoforms generally result from enzyme inhibition or enzyme induction. Enzyme inhibition often involves competition between two active agents for the substrate-binding site of the enzyme, although other mechanisms for inhibition exist. Enzyme induction occurs when an active agent activates an enzyme or stimulates the synthesis of more enzyme protein, enhancing the enzyme's metabolizing capacity.

Cytochrome p450 isozymes identified as important in active agent metabolism are CYP1A2, CYP2A6, CYP2B6, CYP2C9, CYP2C19, CYP2D6, CYP2E1, and CYP3A4. Examples of cytochrome p450 enzymes known to be involved in active agent interactions are the CYP3A subfamily, which is involved in many clinically significant active agent interactions, including those involving non-sedating antihistamines and cisapride, and CYP2D6, which is responsible for the metabolism of many psychotherapeutic agents, such as thioridazine. CYP1A2 and CYP2E1 enzyme are involved in active agent interactions involving theophylline. CYP2C9, CYP1A2, and CYP2C19 are involved in active agent interactions involving warfarin. Phenytoin and fosphenytoin are metabolized by CYP2C9, CYP2C19, and CYP3A4.

Additionally, several cytochrome p450 isozymes are known to be genetically polymorphic, leading to altered substrate metabolizing ability in some individuals. Allelic variants of CYP2D6 are the best characterized, with many resulting in an enzyme with reduced, or no, catalytic activity. Gene duplication also occurs. As a result, four phenotypic subpopulations of metabolizers of CYP2D6 substrates exist: poor (PM), intermediate (IM), extensive (EM), and ultrarapid (UM). The genetic polymorphisms vary depending on the population in question. For example, Caucasian populations contain a large percentage of individuals who are poor metabolizers, due to a deficiency in CYP2D6—perhaps 5-10% of the population, while only 1-2% of Asians are PMs. CYP2C9, which catalyzes the metabolism of a number of commonly used active agents, including that of warfarin and phenytoin, is also polymorphic. The two most common CYP2C9 allelic variants have reduced activity (5-12%) compared to the wild-type enzyme. Genetic polymorphism also occurs in CYP2C19, for which at least 8 allelic variants have been identified that result in catalytically inactive protein. About 3% of Caucasians are poor metabolizers of active agents metabolized by CYP2C19, while 13-23% of Asians are poor metabolizers of active agents metabolized by CYP2C19. Allelic variants of CYP2A6 and CYP2B6 have also been identified as affecting enzyme activity. At least one inactive CYP2A6 variant occurs in Caucasians at a frequency of 1-3%, resulting in a PM phenotype. A whole gene deletion has been identified in a Japanese population, with an allelic frequency of 21%; homozygotes in this mutation show a PM phenotype. For CYP2B6, about 3-4% of Caucasians have a polymorphism producing a PM phenotype.

Several studies, in vitro and in vivo, relating to the metabolism of quinine by particular human cytochrome p450 isozymes have been published; most have focused on establishing the metabolism of quinine using known inhibitors of particular cytochrome p450s. However, Zhao et al. (J. Pharm Exp Ther 1996 279:1327-1334) used human liver microsomes to study which of nine recombinant human cytochrome p450 isoforms (CYP1A1, CYP1A2, CYP2A6, CYP2B6, CYP2C9, CYP2C19, CYP2D6, CYP2E1 and CYP3A4) were involved in the 3-hydroxylation of quinine in humans. Zhao et al. determined that 3-hydroxylation of quinine is mediated mainly by CYP3A4 and to a minor extent by CYP2C19. Further, Zhao et al. used recombinant human CYP3A4 and CYP2C19 singly expressed in human B lymphoblastoid cells (Gentest Corp., Woburn, Mass.) to determine kinetic parameters for 3-hydroxylation of quinine by CYP3A4 (K_{M}=114.4±18.0 (s.d.) μM) and CYP2C19 (K_{M}=46.3±7.8 (s.d.) μM). Similar results were obtained for the mean apparent K_{M} (83±19 (s.d.) μM) for 3-hydroxylation of quinine by CYP3A4 in human liver microsomes by Zhang et al. (Br. J. Clin. Pharmacol. 1997, 43:245-252).

A few studies of the inhibitory effects of quinine on particular human cytochrome p450 isozymes are also published. Quinine and quinidine are well-known inhibitors of CYP2D6 activity. For example, for the debrisoquine 4-hydroxylase activity of CYP2D6 in human liver microsomes, an IC50 of 223 μM has been determined for quinine (Kobayashi, Biochem Pharmacol 1989; 38:2795-2799). Ching et al. (Xenobiotica 2001 31(11):757-67) reported that quinidine and quinine each inhibited CYP1A1 by competitive inhibition with an IC50 of 1-3 μM with substrate concentrations near the K_{M} of catalysis, but showed negligible inhibition of CYP1A2. The inhibition of recombinant CYP1A1 and CYP1A2 activity by quinidine and quinine was evaluated using ethoxyresorutin O-deethylation, phenacetin O-deethylation and propranolol desisopropylation as probe catalytic pathways. Weak inhibition of human CYP2A6 coumarin 7-hydroxylase activity by quinine, with an IC50 value of 160 μm was reported by Hirano et al. (J. Pharm. Pharmacol. 2003 55(12):1667-72).

Furthermore, a few human cytochrome p450 isozymes are reported to be induced by quinine. Bapiro et al. (Eur. J. Clin. Pharmacol. 2002 58(8):537-542) reported that quinine induced human CYP1A1 and CYP1A2 activity and showed that the induction was due to increased mRNA expression levels. Ngui et al. (Drug Met. Disp. 2000 28(9):1043-1050) reported that quinidine and quinine, at concentrations of 20 or 100 μM, enhanced activity of CYP3A4 in human liver microsomes in a reaction producing 5-hydroxy diclofenac from diclofenac by 6- to 9-fold.

By understanding the unique functions and characteristics of Phase I and Phase II metabolic enzymes, such as the cytochrome p450 enzyme superfamily, physicians may better anticipate and manage active agent interactions and may predict or explain an individual's response to a particular therapeutic regimen.

There accordingly remains a need in the art for improved methods for the administration and use of quinine, in particular methods that take into account the effects of quinine on activity of cytochrome P450 isozymes.

### SUMMARY

Disclosed herein are methods of using quinine. Quinine can be used in prevention or treatment of various diseases or conditions, including, for example, malaria caused by Plasmodium species; leg cramps, including for example nocturnal recumbency leg muscle cramps, idiopathic leg cramps, and leg cramps caused by athletic exertion; or babesiosis caused by Babesia microti.

In one embodiment, the method comprises informing a user that quinine is metabolized by cytochrome p450 1A2; an inhibitor of cytochrome p450 1A2, 2B6, 2C8, or 2C9; or an inducer of CYP2A6, CYP2B6, CYP2C9, or CYP2E1.

In another embodiment, the method comprises informing a user that quinine affects activity of CYP2B6, CYP2C8, CYP2C9, or CYP2E1.

In an embodiment, the method comprises informing a user that quinine is not a substrate of CYP2A6, CYP2C9, CYP2D6, or CYP2E1; not an inhibitor of CYP2E1; or not an inducer of CYP2C19.

In another embodiment, the method comprises obtaining quinine from a container associated with published material providing information that quinine is metabolized by cytochrome p450 1A2; an inhibitor of cytochrome p450 1A2, 2B6, 2C8, or 2C9; or an inducer of CYP2A6, CYP2B6, CYP2C9, or CYP2E1.

In yet another embodiment, the method comprises obtaining quinine from a container associated with published material providing information that quinine affects activity of CYP2B6, CYP2C8, CYP2C9, or CYP2E1.

In an embodiment, the method comprises obtaining quinine from a container associated with published material providing information that quinine is not a substrate of CYP2A6, CYP2C9, CYP2D6, or CYP2E1; not an inhibitor of CYP2E1; or not an inducer of CYP2C19.

Also disclosed herein are methods of manufacturing a quinine product.

In one embodiment, the method comprises packaging a quinine dosage form with published material providing information that quinine is metabolized by cytochrome p450 1A2; an inhibitor of cytochrome p450 1A2, 2B6, 2C8, or 2C9; or an inducer of CYP2A6, CYP2B6, CYP2C9, or CYP2E1.

In another embodiment, the method comprises packaging a quinine dosage form with published material providing information that quinine affects activity of CYP2B6, CYP2C8, CYP2C9, or CYP2E1.

In another embodiment, the method comprises packaging a quinine dosage form with published material providing information that quinine is not a substrate of CYP2A6, CYP2C9, CYP2D6, or CYP2E1; not an inhibitor of CYP2E1; or not an inducer of CYP2C19.

Also disclosed herein are articles of manufacture comprising a container containing a dosage form of quinine.

In one embodiment, the container is associated with published material informing that quinine is metabolized by cytochrome p450 1A2; an inhibitor of cytochrome p450 1A2, 2B6, 2C8, or 2C9; or an inducer of CYP2A6, CYP2B6, CYP2C9, or CYP2E1.

In another embodiment, the container is associated with published material informing that quinine affects activity of CYP2B6, CYP2C8, CYP2C9, or CYP2E1.

In another embodiment, the container is associated with published material informing a user that quinine is not a substrate of CYP2A6, CYP2C9, CYP2D6, or CYP2E1; not an inhibitor of CYP2E1; or not an inducer of CYP2C19.

These and other embodiments, advantages and features of the present invention become clear when detailed description and examples are provided in subsequent sections.

### DETAILED DESCRIPTION

Disclosed herein are methods of using quinine and quinine products. Specifically disclosed are methods of using quinine and informing the user of certain information. Such information can include the effects of quinine on the activity of a cytochrome p450 isozyme. With the knowledge of the particular information, the administration of quinine to the patient can be optimized to provide safer use of quinine, while oftentimes reducing or minimizing side effects, adverse events, or interactions with other active agents.

Quinine therapy can be considered optimal when effective plasma levels are reached when required. In addition, peak plasma values (Cₘₐₓ) should be as low as possible so as to reduce the incidence and severity of possible side effects.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”).

An “active agent” means a compound, element, or mixture that when administered to a patient, alone or in combination with another compound, element, or mixture, confers, directly or indirectly, a physiological effect on the patient. The indirect physiological effect may occur via a metabolite or other indirect mechanism. When the active agent is a compound, then salts, solvates (including hydrates) of the free compound or salt, crystalline forms, non-crystalline forms, and any polymorphs of the compound are included. Additionally, compounds other than quinine may contain one or more asymmetric elements such as stereogenic centers, stereogenic axes and the like, e.g., asymmetric carbon atoms, so that the compounds can exist in different stereoisomeric forms. Such compounds other than quinine can be, for example, racemates or optically active forms. For compounds other than quinine with two or more asymmetric elements, these compounds can additionally be mixtures of diastereomers. For compounds other than quinine having asymmetric centers, all optical isomers in pure form or mixtures thereof are encompassed.

“Quinine” (cinchonan-9-ol, 6′-methoxy-, (8a,9R)—) as used herein is inclusive of all pharmaceutically acceptable salt forms, crystalline forms, amorphous forms, polymorphic forms, solvates, and hydrates unless specifically indicated otherwise. As used herein, “quinine sulfate” means cinchonan-9-ol, 6′-methoxy-, (8α,9R)—, sulfate (2:1) or cinchonan-9-ol, 6′-methoxy-, (8α,9R)—, sulfate (2:1) dihydrate unless otherwise indicated.

All forms of quinine or other active agent may be employed either alone or in combination.

“Active agent interaction” refers to a change in the metabolism of an active agent in a patient that can occur with co-administration of a second active agent. A “potential active agent interaction” refers to an active agent interaction between two active agents that is theoretically possible based on knowledge that one of the active agents is metabolized by a given cytochrome p450 isozyme and that the second of the active agents is a substrate, inhibitor, or inducer of that cytochrome p450 isozyme.

“Administering quinine with a substance” or “administering quinine and a substance” means quinine and the substance are administered simultaneously in a single dosage form, administered concomitantly in separate dosage forms, or administered in separate dosage forms separated by some amount of time that is within the time in which both quinine and the substance are within the blood stream of a patient. The quinine and the substance need not be prescribed for a patient by the same medical care worker. The substance or quinine need not require a prescription. Administration of quinine or the substance can occur via any appropriate route, for example, oral tablets, oral capsules, oral liquids, inhalation, injection, suppositories or topical contact.

“Affects” include an increase or decrease in degree, level, or intensity; a change in time of onset or duration; a change in type, kind, or effect, or a combination comprising at least one of the foregoing.

As used herein, “allelic variant” means one of the alternative forms at a genetic locus on a single chromosome. For loci in most of the human genome, a human has two chromosomes, which may carry the same or two different allelic variants.

“Altering the dose of an active agent” can mean tapering off, reducing or increasing the dose of the active agent, ceasing to administer the active agent to the patient, or substituting a second active agent for the active agent.

“Bioavailability” means the extent or rate at which an active agent is absorbed into a living system or is made available at the site of physiological activity. For active agents that are intended to be absorbed into the bloodstream, bioavailability data for a given formulation may provide an estimate of the relative fraction of the administered dose that is absorbed into the systemic circulation. “Bioavailability” can be characterized by one or more pharmacokinetic parameters.

A “dosage form” means a unit of administration of an active agent. Examples of dosage forms include tablets, capsules, injections, suspensions, liquids, emulsions, creams, ointments, suppositories, inhalable forms, transdermal forms, and the like.

The term “effective amount” or “therapeutically effective amount” means an amount effective, when administered to a patient, to provide any therapeutic benefit. A therapeutic benefit may be an amelioration of symptoms, e.g., an amount effective to decrease the symptoms of a malaria, for example uncomplicated P. falciparum malaria. The amount that is “effective” will vary from subject to subject, depending on the age and general condition of the individual, the particular active agent, and the like. Thus, it is not always possible to specify an exact “effective amount.” However, an appropriate “effective” amount in any individual case may be determined by one of ordinary skill in the art using routine experimentation. In certain circumstances a patient may not present symptoms of a condition for which the patient is being treated. A therapeutically effective amount of an active agent may also be an amount sufficient to provide a significant positive effect on any indicium of a disease, disorder, or condition, e.g. an amount sufficient to significantly reduce the severity of uncomplicated P. falciparum malaria. A significant effect on an indicium of a disease, disorder, or condition is statistically significant in a standard parametric test of statistical significance, for example Student's T-test, where p≦0.05. An “effective amount or “therapeutically effective amount” of quinine sulfate may also be an amount of about 2000 mg per day or less, specifically about 1944 mg per day or less, or of any dosage amount approved by a governmental authority such as the US FDA, for use in treatment. In some embodiments amounts of 1944 mg quinine sulfate per day, 324 mg quinine sulfate per unit dosage form, or 648 mg quinine sulfate or less per unit dosage form is an “effective amount” or “therapeutically effective amount” of quinine sulfate.

“Efficacy” means the ability of an active agent administered to a patient to produce a therapeutic effect in the patient.

“Informing” means referring to or providing published material, for example, providing an active agent with published material to a user; or presenting information orally, for example, by presentation at a seminar, conference, or other educational presentation, by conversation between a pharmaceutical sales representative and a medical care worker, or by conversation between a medical care worker and a patient; or demonstrating the intended information to a user for the purpose of comprehension.

A “medical care worker” means a worker in the health care field who may need or utilize information regarding an active agent, including a dosage form thereof, including information on safety, efficacy, dosing, administration, or pharmacokinetics. Examples of medical care workers include physicians, pharmacists, physician's assistants, nurses, aides, caretakers (which can include family members or guardians), emergency medical workers, and veterinarians.

As used herein, an enzyme “metabolizing” a substance means the substance is a substrate of the enzyme, i.e., the enzyme can chemically transform the substance.

A substance having a “narrow therapeutic index” (NTI) means a substance falling within any definition of narrow therapeutic index as promulgated by the U.S. Food and Drug Administration or any successor agency thereof, for example, a substance having a less than 2-fold difference in median lethal dose (LD50) and median effective dose (ED50) values or having a less than 2-fold difference in the minimum toxic concentration and minimum effective concentration in the blood; and for which safe and effective use of the substance requires careful titration and patient monitoring.

“Oral dosage form” includes a dosage form for oral administration.

A “patient” means a human or non-human animal in need of medical treatment. Medical treatment can include treatment of an existing condition, such as a disease or disorder, prophylactic or preventative treatment, or diagnostic treatment. In some embodiments the patient is a human patient.

A “pharmaceutical supplier” means a person (other than a medical care worker), business, charitable organization, governmental organization, or other entity involved in the transfer of active agent, including a dosage form thereof, between entities, for profit or not. Examples of pharmaceutical suppliers include pharmaceutical distributors, pharmaceutical wholesalers, pharmacy chains, pharmacies (online or physical), hospitals, HMOs, supermarkets, the Veterans Administration, or foreign businesses or individuals importing active agent into the United States.

“Pharmacokinetic parameters” describe the in vivo characteristics of an active agent (or surrogate marker for the active agent) over time, such as plasma concentration (C), Cₘᵢₙ, Cₘₐₓ, Cₙ, C₂₄, Tₘₐₓ, and AUC. “Cₘₐₓ” is the measured concentration of the active agent in the plasma at the point of maximum concentration. “Cₘᵢₙ” is the measured concentration of the active agent in the plasma at the point of minimum concentration at steady state. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

“Pharmaceutically acceptable salts” include derivatives of the active agent (e.g. quinine), wherein the parent compound is modified by making acid or base addition salts thereof, and further refers to pharmaceutically acceptable solvates, including hydrates, of such compounds and such salts. Also included are all crystalline, amorphous, and polymorph forms. Examples of pharmaceutically acceptable salts include, but are not limited to, mineral or organic acid addition salts; and the like, and combinations comprising one or more of the foregoing salts. The pharmaceutically acceptable salts include salts, for example, from inorganic or organic acids. For example, acid salts include those derived from inorganic acids such as hydrochloric, hydrobromic, sulfuric, sulfamic, phosphoric, nitric and the like. Pharmaceutically acceptable organic salts includes salts prepared from organic acids such as acetic, trifluoroacetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH2)n-COOH where n is 0-4, and the like. Specific quinine salts include quinine sulfate, quinine hydrochloride, quinine dihydrochloride, and hydrates or solvates thereof.

“Phenotype” means an observable trait of an organism resulting from the interplay of environment and genetics. Examples include apparent rate of metabolism of substrates by a cytochrome p450 isozyme of an organism, such as the “poor metabolizer” (PM) or “ultrarapid metabolizer” (UM) phenotypes identified in humans for metabolism of substrates metabolized by CYP2D6.

“Polymorphism” means the differences in a DNA sequence that occur naturally among different individuals of a population. Single nucleotide substitutions, insertions, and deletions of nucleotides and repetitive sequences (microsatellites) are all examples of a polymorphism.

A “product” or “pharmaceutical product” means a dosage form of an active agent plus published material, and optionally packaging.

“Providing” means giving, administering, selling, distributing, transferring (for profit or not), manufacturing, compounding, or dispensing.

“Published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, an audio recording, or other recording or electronic medium.

As used herein, “quinine therapy” refers to medical treatment of a symptom, disorder, or condition by administration of quinine.

“Safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

A “sensitive plasma concentration profile active agent” means an active agent for which a moderate change in plasma concentration can have a deleterious effect on the prescribed therapeutic intent.

Solid dosage forms of quinine comprise up to about 2000 mg quinine free base, specifically about 83 to about 1614 mg quinine free base, more specifically about 269 to about 538 mg quinine free base, yet more specifically about 216 to about 432 mg quinine free base. Solid dosage forms of quinine sulfate dehydrate comprise up to about 2000 mg quinine sulfate dihydrate, specifically about 100 to about 1944 mg quinine sulfate dihydrate, more specifically about 200 to about 700 mg quinine sulfate dihydrate, yet more specifically about 324 to about 648 mg quinine sulfate dihydrate. In another embodiment, solid dosage forms of quinine comprise about 260 to about 520 mg quinine sulfate dihydrate. In one embodiment, the solid dosage form is an oral dosage form, for example, a tablet. Amounts in dosage forms are given for quinine free base and quinine sulfate dihydrate, however equivalent amounts of other forms of quinine can be used.

A “substance” taken or administered with quinine means a substance that affects the safety, bioavailability, plasma concentration, efficacy, or a combination comprising at least one of the foregoing of quinine or the substance. A “substance” can be an active agent, an herbal supplement, a nutritional supplement, a vitamin, a xenobiotic, or an environmental contaminant.

A substance is a “substrate” of enzyme activity when it can be chemically transformed by action of the enzyme on the substance. “Enzyme activity” refers broadly to the specific activity of the enzyme (i.e., the rate at which the enzyme transforms a substrate per mg or mole of enzyme) as well as the metabolic effect of such transformations. Thus, a substance is an “inhibitor” of enzyme activity when the specific activity or the metabolic effect of the specific activity of the enzyme can be decreased by the presence of the substance, without reference to the precise mechanism of such decrease. For example a substance can be an inhibitor of enzyme activity by competitive, non-competitive, allosteric or other type of enzyme inhibition, by decreasing expression of the enzyme, or other direct or indirect mechanisms. Similarly, a substance is an “inducer” of enzyme activity when the specific activity or the metabolic effect of the specific activity of the enzyme can be increased by the presence of the substance, without reference to the precise mechanism of such increase. For example a substance can be an inducer of enzyme activity by increasing reaction rate, by increasing expression of the enzyme, by allosteric activation or other direct or indirect mechanisms. Any of these effects on enzyme activity can occur at a given concentration of active agent in a single sample, donor, or patient without regard to clinical significance. It is possible for a substance to be a substrate, inhibitor, or inducer of an enzyme activity. For example, the substance can be an inhibitor of enzyme activity by one mechanism and an inducer of enzyme activity by another mechanism. The function (substrate, inhibitor, or inducer) of the substance with respect to activity of an enzyme can depend on environmental conditions.

A “user” means a patient, a medical care worker, or a pharmaceutical supplier.

The cytochrome p450 enzymes are a highly diverse superfamily of enzymes. Each cytochrome p450 enzyme is termed an “isoform” or “isozyme” since each derives from a different gene. Cytochrome p450 enzymes are categorized into families and subfamilies by amino acid sequence similarities. These enzymes are designated by the letters “CYP” followed by an Arabic numeral representing the family, a letter representing the sub-family and another Arabic numeral representing a specific gene (e.g., CYP2D6). Particular isozymes discussed herein are named as per the recommendations of the P450 Gene Superfamily Nomenclature Committee (see e.g., “P450 superfamily: Update on new sequences, gene mapping, accession numbers, and nomenclature” Pharmacogenetics 6, 1-42 1996, part A pp. 1-21.). Herein, the designation for a cytochrome p450 isozyme may encompass the homolog from any species identified as having such an isozyme. For example, CYP1A2 genes are known in at least rat, human, rabbit, hamster, dog, guinea pig, mouse, and chicken and the designation “CYP1A2” includes the CYP1A2 protein from any species known to have a CYP1A2 gene. In some embodiments, the designation for a cytochrome p450 isozyme is the human isozyme.

In one embodiment, CYP1A2 is human CYP1A2 (Entrez Gene ID: 1544; reference protein sequence Genbank NP_{—}000752), and includes any allelic variants. Specifically, CYP1A2 includes any allelic variants included in the list of human CYP1A2 allelic variants maintained by the Human Cytochrome P450 (CYP) Allele Nomenclature Committee; more specifically it includes any of the *1 through *16 alleles. Additional reference amino acid sequences for human CYP1A2 include Genbank AAK25728, AAY26399, AAA35738, AAA52163, AAA52163, AAF13599, AAH67424, AAH67425, AAH67426, AAH67427, AAH67428, AAH67429, AAA52154, AAA52146, CAA77335, P05177, Q6NWU3, Q6NWU5, Q9BXX7, and Q9UK49.

In one embodiment, CYP2A6 is human CYP2A6 (Entrez Gene ID: 1548; reference protein sequence Genbank NP_{—}000753), and includes any CYP2A6 allelic variants. Specifically, CYP2A6 includes any allelic variants included in the list of human CYP2A6 allelic variants maintained by the Human Cytochrome P450 (CYP) Allele Nomenclature Committee; more specifically it includes any of the *1 through *22 alleles. Additional reference amino acid sequences for human CYP2A6 include Genbank AAG45229, AAB40518, AAF13600, AAH96253, AAH96254, AAH96255, AAH96256, AAA52067, CAA32097, CAA32117, P11509, Q13120, and Q4VAU0.

In one embodiment, CYP2B6 is human CYP2B6 (Entrez Gene ID: 1555; reference protein sequence Genbank NP_{—}000758), and includes any CYP2B6 allelic variants. Specifically, CYP2B6 includes any allelic variants included in the list of human CYP2B6 allelic variants maintained by the Human Cytochrome P450 (CYP) Allele Nomenclature Committee; more specifically it includes any of the *1 through *25 alleles. Additional reference amino acid sequences for human CYP2B6 include Genbank AAF32444, AAD25924, ABB84469, AAF13602, AAH67430, AAH67431, AAA52144, P20813, Q6NWU1, Q6NWU2, and Q9UNX8.

In one embodiment, CYP2C8 is human CYP2C8 (Entrez Gene ID: 1558; reference protein sequence Genbank NP_{—}110518), and includes any CYP2C8 allelic variants. Specifically, CYP2B8 includes any allelic variants included in the list of human CYP2C8 allelic variants maintained by the Human Cytochrome P450 (CYP) Allele Nomenclature Committee; more specifically it includes any of the *1 through *10 alleles. Additional reference amino acid sequences for human CYP2C8 include Genbank CAH71307, AAR89907, CAA38578, AAH20596, AAA35739, AAA35740, AAA52160, AAA52161, CAA35915, CAA68550, P10632, Q5VX93, Q8WWB1, and Q9UCZ9.

In one embodiment, CYP2C9 is human CYP2C9 (Entrez Gene ID: 1559; reference protein sequence Genbank NP_{—}000762), and includes any CYP2C9 allelic variants. Specifically, CYP2CP includes any allelic variants included in the list of human CYP2C9 allelic variants maintained by the Human Cytochrome P450 (CYP) Allele Nomenclature Committee; more specifically it includes any of the *1 through *24 alleles. Additional reference amino acid sequences for human CYP2C9 include Genbank CAH71303, AAP88931, AAT94065, AAW83816, AAD13466, AAD13467, AAH20754, AAH70317, BAA00123, AAA52159, AAB23864, P11712, Q5EDC5, Q5VX92, Q61RV8, Q8WW80, Q9UEH3, and Q9UQ59.

In one embodiment, CYP2C19 is human CYP2C19 (Entrez Gene ID: 1557; reference protein sequence Genbank NP_{—}00076), and includes any CYP2C19 allelic variants. Specifically, CYP2C19 includes any allelic variants included in the list of human CYP2C19 allelic variants maintained by the Human Cytochrome P450 (CYP) Allele Nomenclature Committee; more specifically it includes any of the *1 through *21 alleles. Additional reference amino acid sequences for human CYP2C19 include Genbank BAD02827, CAH73444, CAH74068, AAV41877, AAL31347, AAL31348, AAA36660, AAB59426, CAA46778, P33261, Q16743, Q767A3, Q8WZB1, and Q8WZB2.

In one embodiment, CYP2D6 is human CYP2D6 (Entrez Gene ID: 1565; reference protein sequence Genbank NP_{—}000097), and includes any CYP2D6 allelic variants. Specifically, it CYP2D6 includes any allelic variants included in the list of human CYP2D6 allelic variants maintained by the Human Cytochrome P450 (CYP) Allele Nomenclature Committee; more specifically it includes any of the *1 through *58 alleles. Additional reference amino acid sequences for human CYP2D6 include Genbank AAS55001, ABB01370, ABB01371, ABB01372, ABB01373, AAA35737, AAA53500, BAD92729, AAU87043, AAH66877, AAH67432, AAH75023, AAH75024, AAI06758, AAI06759, CAG30316, AAA52153, AAA36403, CAA30807, and P10635.

In one embodiment, CYP2E1 is human CYP2E1 (Entrez Gene ID: 1571; reference protein sequence Genbank NP_{—}000764), and includes any CYP2E1 allelic variants. Specifically, CYP2E1 includes any allelic variants included in the list of human CYP2E1 allelic variants maintained by the Human Cytochrome P450 (CYP) Allele Nomenclature Committee; more specifically it includes any of the *1 through *7 alleles. Additional reference amino acid sequences for human CYP2E1 include Genbank CAH70047, BAA00902, BAA08796, AAA52155, AAD13753, AAF13601, CAI47002, AAH67433, AAH67435, AAZ77710, AAA35743, AAD14267, P05181, Q16868, Q5VZD5, Q6LER5, Q6NWT7, and Q6NWT9.

In one embodiment, CYP3A4 is human CYP3A4 (Entrez Gene ID: 1576; reference protein sequence Genbank NP_{—}059488), and includes any CYP3A4 allelic variants. Specifically, CYP3A4 includes any allelic variants included in the list of human CYP3A4 allelic variants maintained by the Human Cytochrome P450 (CYP) Allele Nomenclature Committee; more specifically it includes any of the *1 through *20 alleles. Additional reference amino acid sequences for human CYP3A4 include Genbank AAF21034, AAG32290, AAG53948, EAL23866, AAF13598, CAD91343, CAD91645, CAD91345, AAH69418, AAI01632, BAA00001, AAA35747, AAA35742, AAA35744, AAA35745, CAA30944, P05184, P08684, Q6GRK0, Q7Z448, Q86SK2, Q86SK3, and Q9BZM0.

The ability of quinine to act as a substrate, inhibitor, or inducer of various cytochrome p450 isozymes was determined in studies described below. A summary of the findings of the studies is provided in Table 1.

TABLE 1
Summary of quinine effects on cytochrome p450 isozymes.

| CYP isozyme | Substrate | Inhibitor | Inducer/Inhibitor |
|---|---|---|---|
| 1A2 | + | + | + |
| 2A6 | 0 | + | + |
| 2B6 | ND | + | + |
| 2C8 | ND | + | A |
| 2C9 | 0 | + | + |
| 2C19 | + | + | 0 |
| 2D6 | 0 | + | − |
| 2E1 | 0 | 0 | + |
| 3A4 | 0 | 0 | + |

For each study to determine a possible function of quinine (i.e., substrate, inhibitor, or inducer), there is a column in the table. A “+” in a particular column and row indicates that the study found that quinine functioned in that capacity with respect to the cytochrome p450 isozyme represented in that row, while a “0” indicates that the results did not support that quinine functioned in that capacity with respect to the cytochrome p450 isozyme represented in that row. In the column labeled Inducer/Inhibitor, a “+” denotes that the quinine functioned as an inducer of the CYP isozyme, while a “−” denotes that quinine functioned as an inhibitor of the CYP isozyme under the conditions of the induction/inhibition study. For example, quinine was found to be a substrate as well as an inhibitor of CYP2C19 activity, an inhibitor of CYP2D6 and an inducer of CYP2E1 activity. The symbol “ND” indicates that no experiment was performed. The symbol “A” indicates the induction/inhibition study results did not permit an unambiguous interpretation of effect.

As summarized in Table 1, quinine was found to be a substrate for CYP1A2 and CYP2C19. Additionally, quinine was determined to be an inhibitor of the cytochrome p450 isozymes CYP1A2, CYP2A6, CYP2B6, CYP2C8, CYP2C9, CYP2C19, and CYP2D6 and also an inducer of the cytochrome p450 isozymes CYP1A2, CYP2A6, CYP2B6, CYP2C9, CYP2E1, and CYP3A4. Quinine was determined not to be a substrate of CYP2A6, CYP2C9, CYP2D6, or CYP2E1. Quinine was also determined not to inhibit CYP2E1 or CYP3A4 and not to induce CYP2C19.

Enzymes involved in Phase I and Phase II active agent metabolism, such as the cytochrome p450 isozymes, respond to the constantly changing types and amounts of substrate active agents they encounter. For example, changes in active agent metabolism due to competition for the same cytochrome p450 isoform can change the clinical effectiveness or safety of an active agent by altering the plasma concentration of the active agent or its metabolite(s). Similarly, inhibition or induction of the cytochrome p450 isoform that metabolizes a particular active agent can change the clinical effectiveness or safety of that active agent. Therefore, for any cytochrome p450 for which quinine acts as a substrate, inhibitor, or inducer, the administration of quinine with a substance that is a substrate, inhibitor, or inducer of that cytochrome p450 can affect the metabolism of the quinine or the substance. For the case in which the substance is a narrow therapeutic index active agent, such as warfarin or phenytoin, too little of the active agent in the blood stream can lead to insufficient therapeutic activity, while a too large dose of the active agent can lead to excessive therapeutic activity or toxicity, both of which can be detrimental.

The invention provides methods of using quinine. These methods include using quinine in the treatment or prevention of various diseases or conditions, including for example, parasitic diseases caused by Plasmodium species (e.g., Plasmodium falciparum, etc.); leg cramps, including for example nocturnal recumbency leg muscle cramps, idiopathic leg cramps, and leg cramps caused by athletic exertion; or babesiosis caused by Babesia microti.

In one embodiment, the method comprises informing a user that quinine is a substrate of cytochrome p450 1A2; an inhibitor of cytochrome p450 1A2, 2B6, 2C8, or 2C9; or an inducer of CYP2A6, CYP2B6, CYP2C9, or CYP2E1. In another embodiment, the method comprises informing a user that quinine affects activity of a cytochrome p450 isozyme. The cytochrome p450 isozyme can be CYP2B6, CYP2C8, CYP2C9, or CYP2E1. In certain embodiments the cytochrome p450 isozyme is a human enzyme. The method can further comprise providing the user with quinine.

Informing the user that quinine affects the activity of a cytochrome p450 isozyme includes providing a user with information about any effect of quinine on the activity of the cytochrome p450 isozyme. Informing the user that quinine affects the activity of a cytochrome p450 isozyme includes informing a user of any of the following: that quinine is a substrate of cytochrome p450 1A2; that quinine is metabolized by CYP1A2; that a cytochrome p450 isozyme metabolizing quinine is CYP1A2; that quinine is an inhibitor of activity of cytochrome p450 1A2, 2B6, 2C8, or 2C9; that quinine is an inducer of activity of CYP2A6, CYP2B6, CYP2C9, or CYP2E1; that there is a potential active agent interaction between quinine and an active agent that is a substrate, inhibitor, or inducer of CYP1A2; that caution is recommended when quinine and a substrate of CYP2A6, CYP2B6, or CYP2C9 are administered to a patient having a poor metabolizer phenotype for or reduced activity of the cytochrome p450 isozyme; that the allelic variants of CYP2A6, CYP2B6, or CYP2C9 present in the patient can further affect a potential active agent interaction between quinine and an active agent; that there is a potential active agent interaction of quinine with an active agent that is a substrate of cytochrome p450 1A2, 2A6, 2B6, 2C8, 2C9, or 2E1; that quinine can induce the metabolism of a substance that is a substrate of CYP2A6, CYP2B6, CYP2C9, or CYP2E1; that caution is recommended when administering quinine with a substance when the substance is an active agent having a sensitive plasma concentration profile or a narrow therapeutic index; that there is a potential active agent interaction of quinine with warfarin; that quinine affects the activity of cytochrome p450 1A2, 2A6, 2B6, 2C8, 2C9, or 2E1; that there is a potential active agent interaction of quinine with a substance that is a substrate of CYP1A2, CYP2A6, CYP2B6, CYP2C8, CYP2C9, or CYP2E1.

The method can further comprise informing the user that administration of quinine with a substance can affect the plasma concentration, bioavailability, safety, efficacy, or a combination comprising at least one of the foregoing of quinine or the substance. In some embodiments, the method further comprises providing the user with the substance.

Informing the user that administration of quinine with a substance can affect the plasma concentration, bioavailability, safety, efficacy, or a combination comprising at least one of the foregoing of quinine or the substance includes providing a user with information about any effect of quinine on plasma concentration, bioavailability, safety, efficacy, or a combination comprising at least one of the foregoing of quinine or the substance. This includes informing a user of any of the following: that taking quinine with an active agent can affect the bioavailability, safety, or efficacy of the active agent or quinine; that administration of quinine and a substance that is a substrate, inhibitor, or inducer of CYP1A2 can affect plasma concentration, bioavailability, safety, efficacy, or a combination comprising at least one of the foregoing of quinine or the substance; that taking quinine with an active agent that is a substrate, inhibitor, or inducer of CYP1A2 can affect the plasma concentration, bioavailability, safety, efficacy, or a combination comprising at least one of the foregoing of quinine or the active agent; that administration of quinine with an active agent that is a cytochrome p450 isozyme substrate having a sensitive plasma concentration profile or a narrow therapeutic index can affect plasma concentration, bioavailability, safety, efficacy, or a combination comprising at least one of the foregoing of the active agent; that quinine can affect the plasma concentration, bioavailability, safety, efficacy, or a combination comprising at least one of the foregoing of an active agent that is a substrate of the cytochrome p450 isozyme; that administration of quinine with an active agent that is a substrate, inhibitor, or inducer of CYP1A2 or that is a substrate of CYP2A6, CYP2B6, CYP2C8, CYP2C9, CYP2C19, CYP2D6, CYP2E1, or CYP3A4 can affect plasma concentration, bioavailability, safety, efficacy, or a combination comprising at least one of the foregoing of the active agent or quinine; that administration of quinine with an active agent that is a CYP2A6, CYP2B6, CYP2C8, CYP2C9, CYP2C19, CYP2D6, CYP2E1, or CYP3A4 substrate having a sensitive plasma concentration profile or a narrow therapeutic index can affect plasma concentration, bioavailability, safety, efficacy, or a combination comprising at least one of the foregoing of the active agent; that administration of quinine with a substance that is metabolized by CYP2A6, CYP2B6, CYP2C9, or CYP2E1 can result in decreased plasma concentration of the substance; or that administration of quinine with a substance that is metabolized by CYP1A2, CYP2B6, CYP2C8, or CYP2C9 can result in increased plasma concentration of the substance.

The effect of administration of quinine with the substance can be determined by comparison of the plasma concentration, bioavailability, safety, efficacy, or a combination comprising at least one of the foregoing of the substance with and without administration of quinine or by comparison of the plasma concentration, bioavailability, safety, efficacy, or a combination comprising at least one of the foregoing of quinine with and without administration of the substance.

In yet another embodiment, the method comprises informing a user that quinine is not a substrate of CYP2A6, CYP2C9, CYP2D6, or CYP2E1; not an inhibitor of CYP2E1; or not an inducer of CYP2C19. The method can further comprise informing the user that interaction of quinine with a substance that is an inhibitor or an inducer of CYP2A6, CYP2C9, CYP2D6, or CYP2E1 is unlikely or that administration of quinine with a substance that is a substrate of CYP2C19 is unlikely to result in reduced plasma concentration of the substance; or that administration of quinine with a substrate of CYP2E1 is unlikely to result in increased plasma concentration of the substance. The method can further comprise providing the user with quinine. In some embodiments, the method further comprises providing the user with the substance.

In another embodiment, the method comprises informing a user that quinine is metabolized by a cytochrome p450 isozyme. The cytochrome p450 isozyme metabolizing quinine is CYP1A2. In some embodiments, the method further comprises informing the user that administration of quinine and a substance that is a substrate, inhibitor, or inducer of CYP1A2 can affect plasma concentration, bioavailability, safety, efficacy, or a combination comprising at least one of the foregoing of quinine or the substance.

The method also comprises informing a user that quinine is an inhibitor of a cytochrome p450 isozyme. Cytochrome p450 isozymes inhibited by quinine include CYP1A2, CYP2B6, CYP2C8, and CYP2C9. The method also comprises informing a user that quinine is an inducer of a cytochrome p450 isozyme. Cytochrome p450 isozymes that are induced by quinine include CYP2A6, CYP2B6, CYP2C9, and CYP2E1. In some embodiments the method further comprises informing a user that administration of quinine and a substance that is a substrate of the cytochrome p450 isozyme can affect plasma concentration, bioavailability, safety, efficacy, or a combination comprising at least one of the foregoing of the substance.

In some embodiments, the method of using quinine can further comprise administering quinine or a substance. Administration may be to a patient by the patient, a medical care worker, or other user. Quinine can be administered in a therapeutically effective amount. The substance can be an active agent. The active agent can have a sensitive plasma concentration profile or a narrow therapeutic index. In some embodiments, the method can further comprise informing the user that caution is recommended when administering quinine with a substance which is an active agent having a sensitive plasma concentration profile or a narrow therapeutic index. The method can also comprise monitoring a patient's plasma concentration of quinine or an active agent as AUC_{0-INF}, AUC₀₋ₜ, C_{MAX}, or a combination of any of the foregoing pharmacokinetic parameters or altering dosing of the active agent or quinine for the patient based on the determined plasma concentration of the active agent or quinine.

In all of the embodiments herein, a medical care worker can determine the plasma concentration of an active agent, including quinine, by performing or ordering the performance of any suitable method. For example, the medical care worker could order a test using blood drawn from the patient for determining the plasma concentration of the active agent.

Medical information provided in any of the methods described herein concerning the effects of administering quinine with an additional substance may alternatively be provided in layman's terms, so as to be better understood by patients or non-medical professionals. Those of skill in the medical art are familiar with the various layman's terms that can be used to describe the effects of active agent interactions.

Additionally, the method can comprise determining the metabolizer phenotype of the patient for a cytochrome p450 isozyme; specifically the cytochrome p450 isozyme is CYP2A6, CYP2B6, CYP2C9, CYP2C19, or CYP2D6. Determining the metabolizer phenotype of the patient can be achieved by determining the allelic variant of the patient for the cytochrome p450 isozyme.

Various laboratory methods are known, including ones that are commercially available, for detecting the presence of allelic variants of cytochrome p450 isozymes in an individual or determining the metabolizer phenotype of an individual for a particular cytochrome p450 isozyme. Any suitable method known in the art may be used. Methods include analyzing a blood sample from the individual to determine the allelic variant of a particular cytochrome p450 isozyme gene present in the individual (for example by genotyping or haplotyping DNA or RNA from the gene using mass spectrometry, gel electrophoresis, or TAQMAN assays; or analyzing the protein sequence expressed by the gene). The metabolizer phenotype of the individual can be inferred based on the known properties of the allelic variants determined to be present in the individual. Alternatively, the blood sample can be used to measure enzyme activity of the cytochrome p450 isozyme using a suitable assay and isozyme-selective substrate. Among suitable isozyme-selective substrates are those used in the studies herein, or those suggested in publications of the United States Food and Drug Administration (FDA) directed to collecting cytochrome p450 isozyme data for regulatory submissions relating to an active agent, for example, the document “Drug Interaction Studies-Study Design, Data Analysis, and Implications For Dosing and Labeling; Preliminary Concept Paper”, dated Oct. 1, 2004, and available from the “Genomics at FDA” regulatory information page of the FDA website.

In yet another embodiment, the method of using quinine comprises obtaining quinine from a container associated with published material providing information that quinine affects activity of a cytochrome p450. Information can also be provided that administering quinine with a substance can affect plasma concentration, bioavailability, safety, efficacy, or a combination comprising at least one of the foregoing of the substance or quinine. The provided information may be any information disclosed herein concerning the effects of quinine or a substance on the activity of a cytochrome p450 isozyme or any information disclosed herein concerning the effects of quinine when administered with a substance on the plasma concentration, bioavailability, safety, efficacy, or a combination comprising at least one of the foregoing of the substance or quinine. The method also comprises providing quinine in the container providing such information. The method can further comprise ingesting the quinine or the substance.

The information provided by the published material can comprise any combination of information disclosed herein concerning the effects of quinine on the activity of a cytochrome p450 isozyme or on the plasma concentration, bioavailability, safety, efficacy, or a combination comprising at least one of the foregoing of quinine or a substance. The information may also comprise any combination of information disclosed herein concerning the effects of a substance on the activity of a cytochrome p450 isozyme or on the plasma concentration, bioavailability, safety, efficacy, or a combination comprising at least one of the foregoing of quinine or a substance when the substance is used with quinine.

The information provided can also be that quinine is not a substrate of CYP2A6, CYP2C9, CYP2D6, or CYP2E1; not an inhibitor of CYP2E1; or not an inducer of CYP2C19; or that interaction of quinine with a substance that is an inhibitor or an inducer of CYP2A6, CYP2C9, CYP2D6, or CYP2E1 is unlikely; or that administration of quinine with a substance that is a substrate of CYP2C19 is unlikely to result in reduced plasma concentration of the substance; or that administration of quinine with a substrate of CYP2E1 is unlikely to result in increased plasma concentration of the substance.

Also disclosed herein are methods of manufacturing a quinine pharmaceutical product.

In one embodiment, the method comprises packaging a quinine dosage form with published material providing information that quinine affects activity of a cytochrome p450 isozyme. The cytochrome p450 isozyme can be CYP1A2, CYP2A6, CYP2B6, CYP2C8, CYP2C9, or CYP2E1. The information may also include any information disclosed herein about the effect of quinine or a substance on the activity of a cytochrome p450 isozyme and any information disclosed herein about the effect of quinine or a substance on the plasma concentration, bioavailability, safety, efficacy, or a combination comprising at least one of the foregoing of quinine or the substance when the substance is used with quinine. The information can also include information that administration of quinine with an active agent having a sensitive plasma concentration profile or a narrow therapeutic index that is a substrate of CYP1A6, CYP2A6, CYP2B6, CYP2C8, CYP2C9, or CYP2E1 can affect plasma concentration, bioavailability, safety, efficacy, or a combination comprising at least one of the foregoing of the active agent.

In an embodiment, the method comprises packaging a quinine dosage form with published material providing information that quinine is metabolized by CYP1A2.

In an embodiment, the method comprises packaging a quinine dosage form with published material providing information that quinine is an inhibitor of a CYP1A2, CYP2B6, CYP2C8, or CYP2C9.

In another embodiment, the method comprises packaging a quinine dosage form with published material providing information that quinine is an inducer of CYP2A6, CYP2B6, CYP2C9, or CYP2E1.

In yet another embodiment, the method comprises packaging a quinine dosage form with published material providing information that quinine is not a substrate of CYP2A6, CYP2C9, CYP2D6, or CYP2E1; not an inhibitor of CYP2E1; or not an inducer of CYP2C19. The published material can provide information that interaction of quinine with a substance that is an inhibitor or an inducer of CYP2A6, CYP2C9, CYP2D6, or CYP2E1 is unlikely; that administration of quinine with a substance that is a substrate of CYP2C19 is unlikely to result in reduced plasma concentration of the substance; or that administration of quinine with a substrate of CYP2E1 is unlikely to result in increased plasma concentration of the substance.

The invention provides articles of manufacture.

In some embodiments, the article of manufacture comprises a container containing a dosage form of quinine.

In one embodiment, the container is associated with published material informing that quinine affects activity of a cytochrome p450 isozyme. The cytochrome p450 isozyme can be CYP1A2, CYP2A6, CYP2B6, CYP2C8, CYP2C9, or CYP2E1. The effect of quinine on the activity of the cytochrome p450 isozyme can be any of the following: that quinine is metabolized by cytochrome p450 1A2; that quinine is an inhibitor of cytochrome p450 1A2, 2B6, 2C8, or 2C9; or that quinine is an inducer of CYP2A6, CYP2B6, CYP2C9, or CYP2E1. The published material can further inform that administration of quinine with a substance that is a substrate, inhibitor, or inducer of the cytochrome p450 isozyme can affect plasma concentration, bioavailability, safety, efficacy, or a combination comprising at least one of the foregoing of quinine or the substance. The substance can be an active agent having a sensitive plasma concentration profile or a narrow therapeutic index, and which is a substrate of the cytochrome p450 isozyme. The published material may be in the form of printed labeling, or in some other form. The published material comprising the article of manufacture may also include any information disclosed herein about the effect of quinine or a substance on the activity of a cytochrome p450 isozyme and any information disclosed herein about the effect of quinine or a substance on the plasma concentration, bioavailability, safety, efficacy, or a combination comprising at least one of the foregoing of quinine or the substance.

In another embodiment, the container is associated with published material that includes information that caution is recommended when administering quinine with the substance, wherein the substance is an active agent that has a sensitive plasma concentration profile or a narrow therapeutic index.

In another embodiment, the container is associated with published material informing a user that quinine is not a substrate of CYP2A6, CYP2C9, CYP2D6, or CYP2E1; not an inhibitor of CYP2E1; or not an inducer of CYP2C19. The published material can provide information that interaction of quinine with a substance that is an inhibitor or an inducer of CYP2A6, CYP2C9, CYP2D6, or CYP2E1 is unlikely; administration of quinine with a substance that is a substrate of CYP2C19 is unlikely to result in reduced plasma concentration of the substance; or administration of quinine with a substrate of CYP2E1 is unlikely to result in increased plasma concentration of the substance.

Also disclosed herein is an article of manufacture comprising packaging material and a dosage form contained within the packaging material, wherein the dosage form comprises, as at least one active ingredient, quinine, and wherein the packaging material comprises a label approved by a regulatory agency for the product. The label may inform that quinine affects activity of a cytochrome p450 isozyme; that a cytochrome p450 isozyme metabolizing quinine is CYP1A2; that quinine is an inhibitor of activity of CYP1A2, CYP2B6, CYP2C8, or CYP2C9; or that quinine is an inducer of activity of CYP2A6, CYP2B6, CYP2C9, or CYP2E1. The label may also inform that quinine is not a substrate of CYP2A6, CYP2C9, CYP2D6, or CYP2E1; not an inhibitor of CYP2E1; or not an inducer of CYP2C19. Examples of regulatory agencies are the US FDA or the European Agency for the Evaluation of Medicinal Products (EMEA).

The invention also includes articles of manufacture in which the substance administered with quinine is phenytoin. In one embodiment, the article of manufacture comprises a container holding a dosage form of quinine associated with published material informing that there is a potential active agent interaction with phenytoin, or that administration of quinine with phenytoin can affect the bioavailability, safety, efficacy or a combination comprising at least one of the foregoing of the phenytoin. The published material may further comprise instructions to monitor the blood levels of phenytoin as AUC₀₋ₜ, AUC_{0-INF}, C_{MAX}, or a combination comprising one or more of the foregoing pharmacokinetic parameters.

In embodiments of the articles of manufacture, the dosage form will typically be contained in a suitable container capable of holding and dispensing the dosage form and which will not significantly interact with the active agent(s) in the dosage form. Further, the container will be in physical relation with the published material. The published material may be associated with the container by any means that maintains physical proximity of the two. By way of example, the container and the published material can both be contained in a packaging material such as a box or plastic shrink wrap. Alternatively, the published material can be bonded to the container, such as with glue that does not obscure the published material, or with other bonding or holding means. Yet another alternative is that the published material is placed within the container with the dosage form.

Someone can also hand the published material to the patient, for example a pharmacist can hand a product insert to a patient in conjunction with dispensing the dosage form. The published material may be a product insert, flyer, brochure, or a packaging material for the dosage form such as a bag, or the like.

In any of the embodiments disclosed herein the published material or information associated with or provided by a container can be contained in any fixed and tangible medium. For example, the information can be part of a leaflet, brochure, or other printed material provided with a container or separate from a container. The information can also take the form of a flyer, advertisement, or the label for marketing the active agent approved by a regulatory agency. The information can also be recorded on a compact disk, DVD or any other recording or electronic medium.

The container can be in the form of bubble or blister pack cards, optionally arranged in a desired order for a particular dosing regimen. Suitable blister packs that can be arranged in a variety of configurations to accommodate a particular dosing regimen are well known in the art or easily ascertained by one of ordinary skill in the art.

Quinine dosage forms existing as liquids, solutions, emulsions, or suspensions can be packaged in a container for convenient dosing of pediatric or geriatric patients. For example, prefilled droppers (such as eye droppers or the like), prefilled syringes, and similar containers housing the liquid, solution, emulsion, or suspension form are contemplated.

The substance used with quinine in the methods and articles of manufactures described herein may have certain effects, direct or indirect, on the activity of a cytochrome p450 enzyme. The substance can be a substrate, inhibitor, or inducer of a Phase I or Phase II metabolic enzyme; specifically, the substance is a substrate, inhibitor, or inducer of a cytochrome p450 isozyme. More specifically, the substance is a substrate of CYP1A2, CYP2A6, CYP2B6, CYP2C8, CYP2C9, or CYP2E1; or an inhibitor or inducer of CYP1A2.

In any of the above methods or articles, the substance can be an active agent.

Examples of active agents that are substrates of CYP1A2 include aminophylline, amitriptyline, caffeine, clomipramine, clozapine, cyclobenzaprine, estradiol, fluvoxamine, haloperidol, imipramine, mexiletine, naproxen, olanzapine, ondansetron, phenacetin, acetaminophen, propranolol, riluzole, ropivacaine, tacrine, theophylline, tizanidine, verapamil, (R)-warfarin, zileuton, and zolmitriptan. Examples of active agents that are inhibitors of CYP1A2 include amiodarone, cimetidine, fluoroquinolones, fluvoxamine, furafylline, interferon, methoxsalen, and mibefradil. Examples of inducers of CYP1A2 include insulin, methyl cholanthrene, modafinil, nafcillin, beta-naphthoflavone, omeprazole, and tobacco.

Examples of substances that are substrates of CYP2A6 include aflatoxin B₁, cotinine, coumarin, 1,7-dimethylxanthine, disulfiram, fadrozole, halothane, losigamone, letrozole, methoxyflurane, nicotine, tobacco-specific nitrosamines, SM-12502, tegafur, and valproic acid.

Examples of active agents that are substrates of CYP2B6 include bupropion, cyclophosphamide, efavirenz, ifosfamide, and methadone.

Examples of active agents that are substrates of CYP2C8 include amodiaquine, cerivastatin, paclitaxel, repaglinide, and torsemide.

Examples of active agents that are substrates of CYP2C9 include diclofenac, ibuprofen, meloxicam, S-naproxen, piroxicam, suprofen, tolbutamide, glipizide, losartan, irbesartan, glyburide (glibenclamide), glipizide, glimepiride, amitriptyline, celecoxib, fluoxetine, fluvastatin, nateglinide, phenytoin, rosiglitazone, tamoxifen, torsemide, and S-warfarin.

Examples of active agents that are substrates of CYP2C19 include the proton pump inhibitors: lansoprazole, omeprazole, pantoprazole, and E-3810; the anti-epileptics: diazepam, phenytoin, fosphenytoin, S-mephenytoin, and phenobarbitone (Phenobarbital); as well as amitriptyline, carisoprodol, citalopram, clomipramine, cyclophosphamide, hexobarbital, imipramine, indomethacin, R-mephobarbital, moclobemide, nelfinavir, nilutamide, primidone, progesterone, proguanil, propranolol, teniposide, and R-warfarin.

Examples of substrates of CYP2E1 include enflurane, halothane, isoflurane, methoxyflurane, sevoflurane; acetaminophen, aniline, benzene, chlorzoxazone, ethanol, N,N-dimethyl formamide, and theophylline.

In any of the embodiments described herein, the substance can be a sensitive plasma concentration profile active agent. Examples of a sensitive plasma concentration profile active agent include cyclophosphamide, efavirenz, fosphenytoin, glimepiride, mexiletine, phenytoin, progesterone, tamoxifen, theophylline, warfarin, and any active agent having a narrow therapeutic index.

In any of the embodiments described herein, the substance can be an active agent having a narrow therapeutic index. Examples of narrow therapeutic index active agents include aprindine, carbamazepine, clindamycin, clonazepam, clonidine, cyclosporine, digitoxin, digoxin, disopyramide, ethinyl estradiol, ethosuximide, fosphenytoin, guanethidine, isoprenaline, lithium, methotrexate, phenobarbital, phenytoin, pimozide, prazosin, primidone, procainamide, quinidine, sulfonylurea compounds (e.g., acetohexamide, glibenclamide, gliclazide, glyclopyramide, tolazamide, tolbutamide), tacrolimus, theophylline compounds (e.g., aminophylline, choline theophylline, diprophylline, proxyphylline, and theophylline), thioridazine, valproic acid, warfarin, and zonisamide.

In another embodiment, the active agent comprises phenytoin. Phenytoin, 5,5-diphenylhydantoin, is an antiepileptic active agent useful in the treatment of epilepsy which is eliminated by metabolism by cytochrome p450 isoforms including CYP1A2, CYP2C9, CYP2C19, and CYP3A4. Phenytoin has a narrow therapeutic index such that too little can lead to insufficient results and excessive phenytoin can lead to phenytoin toxicity. The typical clinically effective serum level is about 10 to about 20 μg/mL. The recommended initial dose is one 100 mg capsule 3 to 4 times per day, with 300 mg/day dose in three divided doses or one single dose per day. The dosing of phenytoin can be individualized according to the patient's sensitivity to the active agent by measuring plasma concentration of phenytoin.

Methods of treating uncomplicated P. falciparum malaria, other forms of malaria, leg cramps, or babesiosis with quinine are provided herein. Such methods include informing a user that quinine affects the activity of a cytochrome p450 isozyme. The method may further include informing the user that administration of quinine with a substance can affect the plasma concentration, bioavailability, safety, efficacy, or a combination comprising at least one of the foregoing of quinine or the substance. The method may also include informing the user of any information disclosed herein about the effect of quinine or the substance on the activity of a cytochrome p450 isozyme and any information disclosed herein about the effect of quinine or the substance on the plasma concentration, bioavailability, safety, efficacy, or a combination comprising at least one of the foregoing of quinine or the substance. Methods of treatment may also include providing a user with quinine or administering quinine to a patient.

Methods of treatment include methods in which the user is a patient and additionally comprising administering quinine and an active agent to the patient. The patient may be, for example, a human patient, a patient in need of treatment of uncomplicated P. falciparum malaria, other forms of malaria, leg cramps, or babesiosis, a patient receiving prophylactic quinine treatment, or a patient undergoing quinine therapy. The amount of quinine administered may be a therapeutically effective amount.

Methods of treatment may additionally include monitoring the patient's plasma concentration of the active agent or quinine as AUC_{0-INF}, AUC₀₋ₜ, C_{MAX}, or a combination of any of the foregoing pharmacokinetic parameters. When quinine is administered together with another active agent, methods of treatment can include determining the plasma concentration of the active agent or quinine and altering dosing of the active agent or quinine for the patient based on the determined plasma concentration of the active agent or quinine.

When the substance administered with quinine is an NTI or sensitive plasma concentration profile active agent, methods using a blood test to monitor plasma levels of the NTI or sensitive plasma concentration profile active agent comprise administering to a patient quinine and the NTI or sensitive plasma concentration profile active agent, and monitoring the blood levels of the NTI or sensitive plasma concentration profile active agent as AUC₀₋ₜ, AUC_{0-INF}, C_{MAX}, or a combination comprising one or more of the foregoing pharmacokinetic parameters. Methods can also include altering dosing of the NTI or sensitive plasma concentration profile active agent for the patient based on the determined plasma concentration of the active agent.

In another embodiment, the substance is phenytoin, and a method using a blood test to monitor plasma levels of phenytoin comprises administering to a patient quinine and phenytoin, and monitoring the blood levels of phenytoin as AUC₀₋ₜ, AUC_{0-INF}, C_{MAX}, or a combination comprising one or more of the foregoing pharmacokinetic parameters.

The invention is further illustrated by the following examples.

### EXAMPLE 1

### Determination of Human Cytochrome p450 Isozymes Using Quinine as a Substrate

The study of this example was performed to determine the metabolism of quinine by human cytochrome p450 isoforms CYP1A2, CYP2A6, CYP2C9, CYP2C19, CYP2D6, CYP2E1, and CYP3A4. Microsomes containing singly expressed human cytochrome p450 (CYP) isoforms were incubated in the presence of quinine sulfate. The metabolism of quinine was evaluated by measuring the disappearance of quinine by high-performance liquid chromatography (HPLC) using fluorescence detection.

Commercially available microsomes from baculovirus-infected insect cells containing singly-expressed recombinant wild-type (*1 allele) human CYP enzymes and cDNA-expressed human cytochrome p450 oxidoreductase [BD SUPERSOMES Enzymes; BD Biosciences Discovery Labware (Woburn, Mass.)] were used. For CYP2A6, CYP2C9, CYP2C19, and CYP2E1, the SUPERSOMES also expressed human cytochrome b5 in addition to human cytochrome p450 oxidoreductase and the human CYP isozyme.

Quinine sulfate stock solutions were prepared in water at 100 times the final concentration used in the incubations. The stock solutions were added to incubation mixtures to obtain final concentrations of 1.5, 5, and 15 μM (corresponding to 487, 1622, and 4866 ng quinine sulfate/mL, respectively), each containing 1% water. All incubations were conducted at 37±1° C. in a shaking water bath with three replicates performed at each quinine sulfate concentration. Incubation mixtures of microsomes (corresponding to 10 pmol p450) and quinine sulfate were prepared in 0.1 M Tris buffer. After a 5-minute pre-incubation, an NADPH regenerating system (NRS) was added to the incubation mixtures to initiate reactions, with a final incubation volume of 0.5 mL. Incubations were continued for 30 minutes, and then terminated by adding an equal volume of methanol. Samples were stored at −70° C. in cryovials and then analyzed for quinine.

Positive controls with a suitable isoform-selective substrate were performed for each CYP isoform to verify metabolic activity of the assay system. Concentration of metabolites formed from CYP isoform-selective substrates in the positive control samples was analyzed using liquid chromatography/mass spectrometry (LC/MS) or HPLC using ultraviolet (UV) detection, as appropriate. A table of the substrate, substrate concentration, solvent, metabolite formed, and metabolite assay method for each CYP isozyme studied is below.

TABLE 2
Isoform-selective substrates for cytochrome p450 isozymes.

| CYP | Isoform-selective | Substrate | | | Metabolite |
|---|---|---|---|---|---|
| isoform | substrate | concentration | Solvent | Metabolite formed | Assay |
| CYP1A2 | Phenacetin | 50 μM | ACN | Acetaminophen | LC/MS |
| CYP2A6 | Coumarin | 8 μM | ACN | 7-hydroxy coumarin | HPLC-UV |
| CYP2C9 | Tolbutamide | 150 μM | ACN | 4′-methylhydroxytolbutamide | LC/MS |
| CYP2C19 | S-Mephenytoin | 50 μM | ACN | 4′-hydroxy mephenytoin | LC/MS |
| CYP2D6 | Dextromethorphan | 5 μM | Water | dextrorphan | LC/MS |
| CYP2E1 | Chlorzoxazone | 50 μM | ACN | 6-hydroxy chlorzoxazone | LC/MS |
| CYP3A4 | Testosterone | 100 μM | ACN | 6β-hydroxy testosterone | HPLC-UV |

Matrix controls were performed to determine the background signal from the matrix components (microsomes (10 pmol p450), 0.1N Tris buffer, 1% water, and NRS). Additionally metabolic negative controls were performed to distinguish potential nonenzymatic metabolism of quinine from p450-mediated metabolism. Incubation mixtures were prepared in 0.1 M Tris buffer with SUPERSOMES (10 pmol P450) and quinine (at each concentration). After a 5-minute pre-incubation, 2% sodium bicarbonate solution was added to the incubation mixtures. Incubation was for 30 minutes at a final volume of 0.5 mL. Matrix and metabolic negative controls were terminated by adding an equal volume of methanol. Analysis of samples for quinine was performed subsequent to storage at −70° C.

Results are presented for each studied human cytochrome p450 isozyme in Tables 3-9.

TABLE 3
Metabolism of Quinine Sulfate byExpressed Recombinant Human CYP1A2

| Quinine Sulfate | Quinine Sulfate Present | | | Percent of Metabolic | |
|---|---|---|---|---|---|
| Concentration | Raw | Adjusted (μM) | | Negative Control | |
| (μM) | (μM) | Individual | Mean ± SD | Individual | Mean ± SD |
| MNC | 0.74309 | 1.49 | 1.49 ± 0.00189 | 100 | 100 ± 0.13 |
| (1.5) | 0.74457 | 1.49 | | 100 | |
| | 0.74485 | 1.49 | | 100 | |
| 1.5 | 0.71675 | 1.43 | 1.46 ± 0.0196 | 96.3 | 97.8 ± 1.31 |
| | 0.73398 | 1.47 | | 98.6 | |
| | 0.73338 | 1.47 | | 98.6 | |
| MNC | 3.14286 | 6.29 | 6.04 ± 0.219 | 104 | 100 ± 3.62 |
| (5) | 2.97264 | 5.95 | | 98.5 | |
| | 2.93884 | 5.88 | | 97.4 | |
| 5 | 2.91527 | 5.83 | 5.82 ± 0.0253 | 96.6 | 96.5 ± 0.419 |
| | 2.89740 | 5.79 | | 96.0 | |
| | 2.92180 | 5.84 | | 96.8 | |
| MNC | 7.38302 | 14.8 | 14.5 ± 0.264 | 102 | 100 ± 1.82 |
| (15) | 7.23224 | 14.5 | | 99.8 | |
| | 7.11958 | 14.2 | | 98.3 | |
| 15 | 7.10917 | 14.2 | 14.4 ± 0.171 | 98.1 | 99.4 ± 1.18 |
| | 7.27632 | 14.6 | | 100 | |
| | 7.22493 | 14.4 | | 99.7 | |
| MXC | 0.02236^{a} | N/A | N/A ± N/A | N/A | N/A ± N/A |
| (0) | 0.00000^{a} | N/A | | N/A | |
| | 0.00000^{a} | N/A | | N/A | |

Abbreviations:
SD, standard deviation;
MNC, metabolic negative control;
MXC, matrix control;
N/A, not applicable.
^{a}The Raw value (μM) was below the lowest concentration on the standard curve (0.1 μM).
Note:
For all calculations above, the resulting values are shown with at least three significant figures for display purposes only.

TABLE 4
Metabolism of Quinine Sulfate by Expressed Recombinant Human CYP2A6

| Quinine | Quinine Sulfate Present | | | Percent of Metabolic | |
|---|---|---|---|---|---|
| Sulfate | Raw | Adjusted (μM) | | Negative Control | |
| Concentration (μM) | (μM) | Individual | Mean ± SD | Individual | Mean ± SD |
| MNC | 0.90084 | 1.80 | 1.74 ± 0.0515 | 103 | 100 ± 2.96 |
| (1.5) | 0.86206 | 1.72 | | 98.9 | |
| | 0.85206 | 1.70 | | 97.8 | |
| 1.5 | 0.87219 | 1.74 | 1.76 ± 0.0173 | 100 | 101 ± 0.990 |
| | 0.88850 | 1.78 | | 102 | |
| | 0.88523 | 1.77 | | 102 | |
| MNC | 3.06756 | 6.14 | 5.94 ± 0.165 | 103 | 100 ± 2.78 |
| (5) | 2.92495 | 5.85 | | 98.4 | |
| | 2.92376 | 5.85 | | 98.4 | |
| 5 | 2.91402 | 5.83 | 5.91 ± 0.0676 | 98.0 | 99.4 ± 1.14 |
| | 2.97544 | 5.95 | | 100 | |
| | 2.96920 | 5.94 | | 99.9 | |
| MNC | 7.94915 | 15.9 | 16.2 ± 0.932 | 97.9 | 100 ± 5.74 |
| (15) | 7.76102 | 15.5 | | 95.6 | |
| | 8.64584 | 17.3 | | 106 | |
| 15 | 7.79178 | 15.6 | 15.5 ± 0.260 | 96.0 | 95.8 ± 1.60 |
| | 7.63692 | 15.3 | | 94.1 | |
| | 7.89496 | 15.8 | | 97.2 | |
| MXC | 0.02583^{a} | N/A | N/A ± N/A | N/A | N/A ± N/A |
| (0) | 0.00000^{a} | N/A | | N/A | |
| | 0.00000^{a} | N/A | | N/A | |

Abbreviations:
SD, standard deviation;
MNC, metabolic negative control;
MXC, matrix control;
N/A, not applicable.
^{a}The Raw value (μM) was below the lowest concentration on the standard curve (0.1 μM).
Note:
For all calculations above, the resulting values are shown with at least three significant figures for display purposes only.

TABLE 5
Metabolism of Quinine Sulfate by Expressed Recombinant Human CYP2C9

| Quinine | | | | | |
|---|---|---|---|---|---|
| Sulfate | Quinine Sulfate Present | | | Percent of Metabolic | |
| Concentration | Raw | Adjusted (μM) | | Negative Control | |
| (μM) | (μM) | Individual | Mean ± SD | Individual | Mean ± SD |
| MNC | 0.80607 | 1.61 | 1.59 ± 0.0203 | 101 | 100 ± 1.28 |
| (1.5) | 0.78847 | 1.58 | | 99.3 | |
| | 0.78856 | 1.58 | | 99.3 | |
| 1.5 | 0.79364 | 1.59 | 1.59 ± 0.00385 | 99.9 | 100 ± 0.242 |
| | 0.79656 | 1.59 | | 100 | |
| | 0.79293 | 1.59 | | 99.8 | |
| MNC | 3.05316 | 6.11 | 6.21 ± 0.0937 | 98.4 | 100 ± 1.51 |
| (5) | 3.11204 | 6.22 | | 100 | |
| | 3.14575 | 6.29 | | 101 | |
| 5 | 3.10173 | 6.20 | 6.24 ± 0.0316 | 99.9 | 100 ± 0.509 |
| | 3.13330 | 6.27 | | 101 | |
| | 3.11845 | 6.24 | | 100 | |
| MNC | 7.68827 | 15.4 | 15.6 ± 0.173 | 98.9 | 100 ± 1.11 |
| (15) | 7.77611 | 15.6 | | 100 | |
| | 7.86084 | 15.7 | | 101 | |
| 15 | 7.68818 | 15.4 | 15.5 ± 0.101 | 98.9 | 99.6 ± 0.652 |
| | 7.75836 | 15.5 | | 99.8 | |
| | 7.78668 | 15.6 | | 100 | |
| MXC | 0.02674^{a} | N/A | N/A ± N/A | N/A | N/A ± N/A |
| (0) | 0.00000^{a} | N/A | | N/A | |
| | 0.00000^{a} | N/A | | N/A | |

Abbreviations:
SD, standard deviation;
MNC, metabolic negative control;
MXC, matrix control;
N/A, not applicable
^{a}The Raw value (μM) was below the lowest concentration on the standard curve (0.1 μM)
Note:
For all calculations above, the resulting values are shown with at least three significant figures for display purposes only.

TABLE 6
Metabolism of Quinine Sulfate by Expressed Recombinant Human CYP2C19

| Quinine Sulfate | Quinine Sulfate Present | | | Percent of Metabolic | |
|---|---|---|---|---|---|
| Concentration | Raw | Adjusted (μM) | | Negative Control | |
| (μM) | (μM) | Individual | Mean ± SD | Individual | Mean ± SD |
| MNC | 0.79333 | 1.59 | 1.600 ± 0.0140 | 99.2 | 100 ± 0.876 |
| (1.5) | 0.79912 | 1.60 | | 99.9 | |
| | 0.80727 | 1.61 | | 101 | |
| 1.5 | 0.69376 | 1.39 | 1.38 ± 0.00619 | 86.7 | 86.3 ± 0.387 |
| | 0.68949 | 1.38 | | 86.2 | |
| | 0.68774 | 1.38 | | 86.0 | |
| MNC | 3.00919 | 6.02 | 6.09 ± 0.0964 | 98.9 | 100 ± 1.58 |
| (5) | 3.09870 | 6.20 | | 102 | |
| | 3.02296 | 6.05 | | 99.3 | |
| 5 | 2.81674 | 5.63 | 5.70 ± 0.0695 | 92.5 | 93.6 ± 1.14 |
| | 2.84181 | 5.68 | | 93.4 | |
| | 2.88545 | 5.77 | | 94.8 | |
| MNC | 7.81883 | 15.6 | 15.9 ± 0.211 | 98.5 | 100 ± 1.33 |
| (15) | 8.02199 | 16.0 | | 101 | |
| | 7.97022 | 15.9 | | 100 | |
| 15 | 7.73983 | 15.5 | 18.8 ± 5.44 | 97.5 | 119 ± 34.3 |
| | 12.56125 | 25.1 | | 158 | |
| | 7.96955 | 15.9 | | 100 | |
| MXC | 0.02182^{a} | N/A | N/A ± N/A | N/A | N/A ± N/A |
| (0) | 0.00000^{a} | N/A | | N/A | |
| | 0.00000^{a} | N/A | | N/A | |

Abbreviations:
SD, standard deviation;
MNC, metabolic negative control;
MXC, matrix control;
N/A, not applicable
^{a}The Raw value (μM) was below the lowest concentration on the standard curve (0.1 μM)
Note:
For all calculations above, the resulting values are shown with at least three significant figures for display purposes only.

TABLE 7
Metabolism of Quinine Sulfate by Expressed Recombinant Human CYP2D6

| | Quinine Sulfate Present | | | Percent of Metabolic | |
|---|---|---|---|---|---|
| Quinine Sulfate | Raw | Adjusted (μM) | | Negative Control | |
| Concentration (μM) | (μM) | Individual | Mean ± SD | Individual | Mean ± SD |
| MNC | 0.78801 | 1.58 | 1.56 ± 0.0162 | 101 | 100 ± 1.04 |
| (1.5) | 0.77674 | 1.55 | | 99.7 | |
| | 0.77234 | 1.54 | | 99.1 | |
| 1.5 | 0.76987 | 1.54 | 1.55 ± 0.0140 | 98.8 | 99.2 ± 0.901 |
| | 0.76828 | 1.54 | | 98.6 | |
| | 0.78115 | 1.56 | | 100 | |
| MNC | 3.03103 | 6.06 | 6.03 ± 0.0287 | 101 | 100 ± 0.476 |
| (5) | 3.00732 | 6.01 | | 99.8 | |
| | 3.00517 | 6.01 | | 99.7 | |
| 5 | 3.02711 | 6.05 | 5.99 ± 0.0581 | 100 | 99.3 ± 0.963 |
| | 2.98221 | 5.96 | | 98.9 | |
| | 2.97278 | 5.95 | | 98.6 | |
| MNC | 8.59055 | 17.2 | 17.3 ± 0.255 | 99.3 | 100 ± 1.48 |
| (15) | 8.80138 | 17.6 | | 102 | |
| | 8.57125 | 17.1 | | 99.0 | |
| 15 | 8.57640 | 17.2 | 17.0 ± 0.226 | 99.1 | 98.4 ± 1.31 |
| | 8.59132 | 17.2 | | 99.3 | |
| | 8.38866 | 16.8 | | 96.9 | |
| MXC | 0.02260^{a} | N/A | N/A ± N/A | N/A | N/A ± N/A |
| (0) | 0.00000^{a} | N/A | | N/A | |
| | 0.00000^{a} | N/A | | N/A | |

Abbreviations:
SD, standard deviation;
MNC, metabolic negative control;
MXC, matrix control;
N/A, not applicable
^{a}The Raw value (μM) was below the lowest concentration on the standard curve (0.1 μM)
Note:
For all calculations above, the resulting values are shown with at least three significant figures for display purposes only.

TABLE 8
Metabolism of Quinine Sulfate by Expressed Recombinant Human CYP2E1

| | Quinine Sulfate Present | | | Percent of Metabolic | |
|---|---|---|---|---|---|
| Quinine Sulfate | Raw | Adjusted (μM) | | Negative Control | |
| Concentration (μM) | (μM) | Individual | Mean ± SD | Individual | Mean ± SD |
| MNC | 0.79405 | 1.59 | 1.56 ± 0.0254 | 102 | 100 ± 1.63 |
| (1.5) | 0.76919 | 1.54 | | 98.6 | |
| | 0.77726 | 1.55 | | 99.6 | |
| 1.5 | 0.79005 | 1.58 | 1.57 ± 0.0108 | 101 | 101 ± 0.692 |
| | 0.78006 | 1.56 | | 100 | |
| | 0.78861 | 1.58 | | 101 | |
| MNC | 3.13784 | 6.28 | 6.29 ± 0.0189 | 99.7 | 100 ± 0.301 |
| (5) | 3.14613 | 6.29 | | 100 | |
| | 3.15671 | 6.31 | | 100 | |
| 5 | 3.19023 | 6.38 | 6.37 ± 0.0123 | 101 | 101 ± 0.196 |
| | 3.17827 | 6.36 | | 101 | |
| | 3.18677 | 6.37 | | 101 | |
| MNC | 8.26106 | 16.5 | 16.6 ± 0.0843 | 99.4 | 100 ± 0.508 |
| (15) | 8.33238 | 16.7 | | 100 | |
| | 8.33572 | 16.7 | | 100 | |
| 15 | 8.33386 | 16.7 | 16.6 ± 0.0690 | 100 | 99.8 ± 0.415 |
| | 8.27662 | 16.6 | | 99.6 | |
| | 8.27191 | 16.5 | | 99.5 | |
| MXC | 0.02317^{a} | N/A | N/A ± N/A | N/A | N/A ± N/A |
| (0) | 0.00000^{a} | N/A | | N/A | |
| | 0.00000^{a} | N/A | | N/A | |

Abbreviations:
SD, standard deviation;
MNC, metabolic negative control;
MXC, matrix control;
N/A, not applicable
^{a}The Raw value (μM) was below the lowest concentration on the standard curve (0.1 μM)
Note:
For all calculations above, the resulting values are shown with at least three significant figures for display purposes only.

TABLE 9
Metabolism of Quinine Sulfate by Expressed Recombinant Human CYP3A4

| Quinine Sulfate | Quinine Sulfate Present | | | Percent of Metabolic | |
|---|---|---|---|---|---|
| Concentration | Raw | Adjusted (μM) | | Negative Control | |
| (μM) | (μM) | Individual | Mean ± SD | Individual | Mean ± SD |
| MNC | 0.77637 | 1.55 | 1.56 ± 0.00605 | 99.6 | 100 ± 0.388 |
| (1.5) | 0.78238 | 1.56 | | 100 | |
| | 0.77998 | 1.56 | | 100 | |
| 1.5 | 0.77170 | 1.54 | 1.55 ± 0.0145 | 99.0 | 99.4 ± 0.928 |
| | 0.77008 | 1.54 | | 98.8 | |
| | 0.78334 | 1.57 | | 100 | |
| MNC | 3.12387 | 6.25 | 6.71 ± 0.851 | 93.2 | 100 ± 12.7 |
| (5) | 3.09172 | 6.18 | | 92.2 | |
| | 3.84434 | 7.69 | | 115 | |
| 5 | 3.30505 | 6.61 | 6.35 ± 0.228 | 98.6 | 94.6 ± 3.41 |
| | 3.11418 | 6.23 | | 92.9 | |
| | 3.10094 | 6.20 | | 92.5 | |
| MNC | 8.40508 | 16.8 | 16.3 ± 0.437 | 103 | 100 ± 2.68 |
| (15) | 7.97055 | 15.9 | | 97.5 | |
| | 8.14392 | 16.3 | | 99.6 | |
| 15 | 8.11148 | 16.2 | 16.5 ± 0.254 | 99.2 | 101 ± 1.55 |
| | 8.36346 | 16.7 | | 102 | |
| | 8.26557 | 16.5 | | 101 | |
| MXC | 0.00000^{a} | N/A | N/A ± N/A | N/A | N/A ± N/A |
| (0) | 0.00000^{a} | N/A | | N/A | |
| | 0.00000^{a} | N/A | | N/A | |

Abbreviations:
SD, standard deviation;
MNC, metabolic negative control;
MXC, matrix control;
N/A, not applicable
^{a}The Raw value (μM) was below the lowest concentration on the standard curve (0.1 μM)
Note:
For all calculations above, the resulting values are shown with at least three significant figures for display purposes only.

Table 3 shows the results for recombinant human CYP1A2. Disappearance of quinine was detected following incubation at 1.5 μM with CYP1A2 in the presence of the NADPH-regenerating system at a statistically significant level using an unpaired two-tailed t-test (p≦0.05). The apparent disappearance of quinine sulfate at 5 and 15 μM was not statistically significant (p>0.05; unpaired two-tail t test). These results indicate that quinine is a substrate for the enzymatic activity of CYP1A2.

Table 6 shows the results for recombinant human CYP2C19. In the experiments with CYP2C19, quinine disappearance was evident following incubation with quinine at 1.5 and 5 μM (Table 6). At both these concentrations of quinine, the reduction in the mean amount of quinine from the value for the corresponding metabolic negative controls was statistically significant (p≦0.05) using an unpaired two-tailed t-test. The amount of the disappearance of quinine observed at 15 μM was not statistically significant (p>0.05) compared to the mean values for the corresponding metabolic negative control using a two-tailed t-test. These results indicate that quinine sulfate is a substrate for the enzymatic activity of CYP2C19.

Experiments with the other tested cytochrome p450 isozymes (Tables 4-5 and 8-9) failed to show any statistically significant disappearance of quinine following incubation at the standard conditions, indicating that, within the limits of detection for these experiments, quinine was not used as a substrate by the other tested cytochrome p450 isozymes: CYP2A6, CYP2C9, CYP2D6, and CYP2E1. In these experiments, the quinine sulfate concentration range tested did not yield evidence of metabolism of quinine by the enzyme CYP3A4. Based on the previously determined values of the K_{M} of quinine for CYP3A4, the lack of turnover observed in these experiments at quinine concentrations of 30 μM or less is not unexpected.

### EXAMPLE 2

### Quinine Sulfate Inhibition of Cytochrome p450 Isozymes in Human Microsomes

The study of this example was performed to determine the potential of quinine to inhibit the activities of cytochrome p450 isoforms CYP1A2, CYP2A6, CYP2B6, CYP2C8, CYP2C9, CYP2C19, CYP2D6, CYP2E1, and CYP3A4 in human liver microsomes. Human liver microsomes were incubated in the presence of quinine sulfate and a substrate selective for each CYP isoform. A table of the substrate, substrate concentration, solvent, metabolite formed and metabolite assay method for each CYP isozyme studied is below.

TABLE 10
Isoform-selective substrates for cytochrome p450 isozymes.

| CYP | Isoform-selective | Substrate | | | | Metabolite |
|---|---|---|---|---|---|---|
| isoform | substrate | concentration | | Solvent | Metabolite formed | Assay |
| CYP1A2 | Phenacetin | 50 | μM | ACN | acetaminophen | LC/MS |
| CYP2A6 | Coumarin | 8 | μM | ACN | 7-hydroxycoumarin | HPLC-UV |
| CYP2B6 | S-Mephenytoin | 1 | mM | ACN | nirvanol | LC/MS |
| CYP2C8 | Paclitaxel | 5 | μM | ACN | 6-hydroxypaclitaxel | HPLC-UV |
| CYP2C9 | Tolbutamide | 150 | μM | ACN | 4′-methylhydroxytolbutamide | LC/MS |
| CYP2C19 | S-Mephenytoin | 50 | μM | ACN | 4′-hydroxymephenytoin | LC/MS |
| CYP2D6 | Dextromethorphan | 5 | μM | Water | dextrorphan | LC/MS |
| CYP2E1 | Chlorzoxazone | 50 | μM | ACN | 6-hydroxychlorzoxazone | LC/MS |
| CYP3A4 | Testosterone | 100 | μM | ACN | 6β-hydroxytestosterone | HPLC-UV |

Quinine sulfate stock solutions were prepared in water at 50 times the final concentration and added to incubation mixtures to obtain final concentrations of 0.2, 2, 10, 20, and 30 μM (corresponding to 64.9, 649, 3240, 6490 and 9730 ng quinine sulfate/mL, respectively), each containing 2% water and 1% acetonitrile.

Microsomes were prepared by differential centrifugation of liver homogenates pooled from at least ten human donors.

Incubation mixtures were prepared in 0.1 M Tris buffer and contained microsomes (0.25 mg protein/mL for CYP2C9, CYP2D6, CYP2E1, and CYP3A4; 0.5 mg protein/mL for CYP1A2, CYP2A6, CYP2B6, CYP2C8, and CYP2C19), quinine sulfate, and a CYP isoform-selective substrate. All quinine sulfate incubations were conducted at 37±1° C. in a shaking water bath. After a 5 minute preincubation, NADPH regenerating system (NRS) was added to initiate the reaction. CYP2A6 and CYP3A4 incubations were for 10 minutes. All other incubations were for 30 minutes.

Incubations for CYP2C8 were terminated by adding 1.0 mL of ACN, while all other incubations were terminated by adding 1.0 mL of methanol. Samples were transferred to cryovials and analyzed for metabolite after storage at −70° C. Three replicates were performed at each concentration of quinine sulfate for each cytochrome p450 isozyme.

To verify that the test system was responsive to inhibitors, a positive control using ketoconazole, a selective inhibitor of CYP3A4, was added to a microsome incubation. Four replicates were performed. The test system was considered responsive to inhibitors since the mean specific activity of CYP3A4 in the positive control samples treated with ketoconazole was <14% of the mean specific activity in the corresponding vehicle control samples.

Vehicle control experiments were performed to establish a baseline value for enzyme activity. Incubation mixtures without added quinine sulfate were prepared in 0.1 M Tris buffer with microsomes (0.25 mg protein/mL for CYP2C9, CYP2D6, CYP2E1, and CYP3A4; 0.5 mg protein/mL for CYP1A2, CYP2A6, CYP2B6, CYP2C8, and CYP2C19), 1% ACN, and a CYP isoform-selective substrate. Four replicates were performed.

Quinine sulfate interference control samples were also included to eliminate the possibility of interference by quinine sulfate or its metabolites in detection of the metabolite formed from the isoform-selective substrate. Incubation mixtures containing microsomes (0.25 mg protein/mL for CYP2C9, CYP2D6, CYP2E1, and CYP3A4; 0.5 mg protein/mL for CYP1A2, CYP2A6, CYP2B6, CYP2C8, and CYP2C19), 100 μM quinine sulfate, and 1% substrate solvent were prepared in 0.1 M Tris buffer. Two replicates of the interference control experiments were performed. No interference was detected in any of the metabolite assay methods used.

Results for each CYP isoform, in the presence and absence of quinine sulfate, are reported in Tables 11-19.

TABLE 11
Quinine Sulfate Effects on CYP1A2 Activity in Pooled Human Liver Microsomes

| | Acetaminophen formation | | | Specific Activity | | |
|---|---|---|---|---|---|---|
| Quinine Sulfate | Raw | Adjusted (μM) | | (pmol/min/mg protein) | | Percent |
| (μM) | (μM) | Individual | Mean ± SD | Individual | Mean ± SD | of VC |
| 0 | 0.18081 | 0.181 | 0.170 ± 0.00895 | 24.1 | 22.7 ± 1.19 | 100 |
| (VC) | 0.16106 | 0.161 | | 21.5 | | |
| | 0.16476 | 0.165 | | 22.0 | | |
| | 0.17399 | 0.174 | | 23.2 | | |
| 0.2 | 0.16062 | 0.161 | 0.174 ± 0.0123 | 21.4 | 23.2 ± 1.64 | 102 |
| | 0.18479 | 0.185 | | 24.6 | | |
| | 0.17681 | 0.177 | | 23.6 | | |
| 2 | 0.15504 | 0.155 | 0.160 ± 0.00494 | 20.7 | 21.4 ± 0.659 | 94.1 |
| | 0.16490 | 0.165 | | 22.0 | | |
| | 0.16054 | 0.161 | | 21.4 | | |
| 10 | 0.14709 | 0.147 | 0.149 ± 0.00867 | 19.6 | 19.8 ± 1.16 | 87.4 |
| | 0.14096 | 0.141 | | 18.8 | | |
| | 0.15807 | 0.158 | | 21.1 | | |
| 20 | 0.14179 | 0.142 | 0.144 ± 0.00540 | 18.9 | 19.2 ± 0.721 | 84.7 |
| | 0.15026 | 0.150 | | 20.0 | | |
| | 0.14021 | 0.140 | | 18.7 | | |
| 30 | 0.15139 | 0.151 | 0.149 ± 0.00252 | 20.2 | 19.9 ± 0.336 | 87.6 |
| | 0.14943 | 0.149 | | 19.9 | | |
| | 0.14639 | 0.146 | | 19.5 | | |

Abbreviations:
SD, standard deviation;
VC, vehicle control (2% Water/1% Acetonitrile)
Note:
For all calculations above, the resulting values are shown with at least three significant figures for display purposes only.

TABLE 12
Quinine Sulfate Effects on CYP2A6 Activity in Pooled Human Liver Microsomes

| | 7-Hydrxoycoumarin formation | | | Specific Activity | | |
|---|---|---|---|---|---|---|
| Quinine Sulfate | Raw | Adjusted (μM) | | (pmol/min/mg protein) | | Percent |
| (μM) | (μM) | Individual | Mean ± SD | Individual | Mean ± SD | of VC |
| 0 | 0.47823 | 0.478 | 0.478 ± 0.00615 | 191 | 191 ± 2.46 | 100 |
| (VC) | 0.48398 | 0.484 | | 194 | | |
| | 0.46987 | 0.470 | | 188 | | |
| | 0.48148 | 0.481 | | 193 | | |
| 0.2 | 0.44870 | 0.449 | 0.457 ± 0.00718 | 179 | 183 ± 2.87 | 95.5 |
| | 0.46062 | 0.461 | | 184 | | |
| | 0.46159 | 0.462 | | 185 | | |
| 2 | 0.45106 | 0.451 | 0.456 ± 0.00597 | 180 | 183 ± 2.39 | 95.4 |
| | 0.45537 | 0.455 | | 182 | | |
| | 0.46286 | 0.463 | | 185 | | |
| 10 | 0.42268 | 0.423 | 0.417 ± 0.00604 | 169 | 167 ± 2.42 | 87.1 |
| | 0.41723 | 0.417 | | 167 | | |
| | 0.41062 | 0.411 | | 164 | | |
| 20 | 0.40549 | 0.405 | 0.407 ± 0.00359 | 162 | 163 ± 1.44 | 85.2 |
| | 0.40514 | 0.405 | | 162 | | |
| | 0.41153 | 0.412 | | 165 | | |
| 30 | 0.43524 | 0.435 | 0.433 ± 0.00221 | 174 | 173 ± 0.883 | 90.4 |
| | 0.43132 | 0.431 | | 173 | | |
| | 0.43152 | 0.432 | | 173 | | |

Abbreviations:
SD, standard deviation;
VC, vehicle control (2% Water/1% Acetonitrile)
Note:
For all calculations above, the resulting values are shown with at least three significant figures for display purposes only.

TABLE 13
Quinine Sulfate Effects on CYP2B6 Activity in Pooled Human Liver Microsomes

| | Nirvanol formation | | | Specific Activity | | |
|---|---|---|---|---|---|---|
| Quinine Sulfate | Raw | Adjusted (μM) | | (pmol/min/mg protein) | | Percent |
| (μM) | (μM) | Individual | Mean ± SD | Individual | Mean ± SD | of VC |
| 0 | 0.22798 | 0.228 | 0.220 ± 0.0102 | 30.4 | 29.3 ± 1.36 | 100 |
| (VC) | 0.20525 | 0.205 | | 27.4 | | |
| | 0.22541 | 0.225 | | 30.1 | | |
| | 0.22126 | 0.221 | | 29.5 | | |
| 0.2 | 0.21689 | 0.217 | 0.212 ± 0.0117 | 28.9 | 28.3 ± 1.56 | 96.3 |
| | 0.19853 | 0.199 | | 26.5 | | |
| | 0.22036 | 0.220 | | 29.4 | | |
| 2 | 0.21610 | 0.216 | 0.203 ± 0.0118 | 28.8 | 27.0 ± 1.58 | 92.2 |
| | 0.19362 | 0.194 | | 25.8 | | |
| | 0.19848 | 0.198 | | 26.5 | | |
| 10 | 0.16712 | 0.167 | 0.173 ± 0.00723 | 22.3 | 23.0 ± 0.964 | 78.5 |
| | 0.18092 | 0.181 | | 24.1 | | |
| | 0.17026 | 0.170 | | 22.7 | | |
| 20 | 0.15344 | 0.153 | 0.160 ± 0.0112 | 20.5 | 21.3 ± 1.50 | 72.6 |
| | 0.17275 | 0.173 | | 23.0 | | |
| | 0.15316 | 0.153 | | 20.4 | | |
| 30 | 0.15832 | 0.158 | 0.161 ± 0.00712 | 21.1 | 21.5 ± 0.950 | 73.4 |
| | 0.16954 | 0.170 | | 22.6 | | |
| | 0.15633 | 0.156 | | 20.8 | | |

Abbreviations:
SD, standard deviation;
VC, vehicle control (2% Water/1% Acetonitrile)
Note:
For all calculations above, the resulting values are shown with at least three significant figures for display purposes only.

TABLE 14
Quinine Sulfate Effects on CYP2C8 Activity in Pooled Human Liver Microsomes

| | 6-Hydroxypaclitaxel formation | | | Specific Activity | | |
|---|---|---|---|---|---|---|
| Quinine Sulfate | Raw | Adjusted (μM) | | (pmol/min/mg protein) | | Percent |
| (μM) | (μM) | Individual | Mean ± SD | Individual | Mean ± SD | of VC |
| 0 | 0.15139 | 0.151 | 0.152 ± 0.00195 | 20.2 | 20.3 ± 0.260 | 100 |
| (VC) | 0.15431 | 0.154 | | 20.6 | | |
| | 0.14992 | 0.150 | | 20.0 | | |
| | 0.15326 | 0.153 | | 20.4 | | |
| 0.2 | 0.16755 | 0.168 | 0.168 ± 0.00985 | 22.3 | 22.5 ± 1.31 | 111 |
| | 0.15897 | 0.159 | | 21.2 | | |
| | 0.17861 | 0.179 | | 23.8 | | |
| 2 | 0.14232 | 0.142 | 0.142 ± 0.00123 | 19.0 | 18.9 ± 0.164 | 93.0 |
| | 0.14220 | 0.142 | | 19.0 | | |
| | 0.14013 | 0.140 | | 18.7 | | |
| 10 | 0.12015 | 0.120 | 0.121 ± 0.00326 | 16.0 | 16.1 ± 0.434 | 79.3 |
| | 0.12414 | 0.124 | | 16.6 | | |
| | 0.11769 | 0.118 | | 15.7 | | |
| 20 | 0.09035 | 0.0904 | 0.0872 ± 0.00368 | 12.0 | 11.6 ± 0.491 | 57.3 |
| | 0.08813 | 0.0881 | | 11.8 | | |
| | 0.08316 | 0.0832 | | 11.1 | | |
| 30 | 0.06905 | 0.0691 | 0.0744 ± 0.00467 | 9.21 | 9.92 ± 0.622 | 48.9 |
| | 0.07642 | 0.0764 | | 10.2 | | |
| | 0.07770 | 0.0777 | | 10.4 | | |

Abbreviations:
SD, standard deviation;
VC, vehicle control (2% Water/1% Acetonitrile)
Note:
For all calculations above, the resulting values are shown with at least three significant figures for display purposes only.

TABLE 15
Quinine Sulfate Effects on CYP2C9 Activity in Pooled Human Liver Microsomes

| | 4′-Methylhydroxytolbutamide formation | | | Specific Activity | | |
|---|---|---|---|---|---|---|
| Quinine Sulfate | Raw | Adjusted (μM) | | (pmol/min/mg protein) | | Percent |
| (μM) | (μM) | Individual | Mean ± SD | Individual | Mean ± SD | of VC |
| 0 | 0.32381 | 0.324 | 0.323 ± 0.0168 | 86.3 | 86.0 ± 4.48 | 100 |
| (VC) | 0.34590 | 0.346 | | 92.2 | | |
| | 0.31170 | 0.312 | | 83.1 | | |
| | 0.30911 | 0.309 | | 82.4 | | |
| 0.2 | 0.33427 | 0.334 | 0.336 ± 0.00280 | 89.1 | 89.6 ± 0.746 | 104 |
| | 0.33931 | 0.339 | | 90.5 | | |
| | 0.33469 | 0.335 | | 89.3 | | |
| 2 | 0.32604 | 0.326 | 0.322 ± 0.0220 | 86.9 | 85.8 ± 5.87 | 99.7 |
| | 0.34138 | 0.341 | | 91.0 | | |
| | 0.29797 | 0.298 | | 79.5 | | |
| 10 | 0.30932 | 0.309 | 0.305 ± 0.0113 | 82.5 | 81.4 ± 3.02 | 94.6 |
| | 0.31372 | 0.314 | | 83.7 | | |
| | 0.29229 | 0.292 | | 77.9 | | |
| 20 | 0.28857 | 0.289 | 0.295 ± 0.00682 | 77.0 | 78.8 ± 1.82 | 91.5 |
| | 0.30220 | 0.302 | | 80.6 | | |
| | 0.29520 | 0.295 | | 78.7 | | |
| 30 | 0.26259 | 0.263 | 0.286 ± 0.0206 | 70.0 | 76.2 ± 5.50 | 88.6 |
| | 0.29241 | 0.292 | | 78.0 | | |
| | 0.30218 | 0.302 | | 80.6 | | |

Abbreviations:
SD, standard deviation;
VC, vehicle control (2% Water/1% Acetonitrile)
Note:
For all calculations above, the resulting values are shown with at least three significant figures for display purposes only.

TABLE 16
Quinine Sulfate Effects on CYP2C19 Activity in Pooled Human Liver Microsomes

| | 4′-Hydroxymephenytoin formation | | | Specific Activity | | |
|---|---|---|---|---|---|---|
| Quinine Sulfate | Raw | Adjusted (μM) | | (pmol/min/mg protein) | | Percent |
| (μM) | (μM) | Individual | Mean ± SD | Individual | Mean ± SD | of VC |
| 0 | 0.10297 | 0.103 | 0.0997 ± 0.00470 | 13.7 | 13.3 ± 0.626 | 100 |
| (VC) | 0.09283 | 0.0928 | | 12.4 | | |
| | 0.10247 | 0.102 | | 13.7 | | |
| | 0.10050 | 0.101 | | 13.4 | | |
| 0.2 | 0.10819 | 0.108 | 0.0988 ± 0.00846 | 14.4 | 13.2 ± 1.13 | 99.1 |
| | 0.09176 | 0.0918 | | 12.2 | | |
| | 0.09649 | 0.0965 | | 12.9 | | |
| 2 | 0.10239 | 0.102 | 0.102 ± 0.00606 | 13.7 | 13.6 ± 0.807 | 102 |
| | 0.10780 | 0.108 | | 14.4 | | |
| | 0.09571 | 0.0957 | | 12.8 | | |
| 10 | 0.10472 | 0.105 | 0.0971 ± 0.00697 | 14.0 | 13.0 ± 0.929 | 97.4 |
| | 0.09103 | 0.0910 | | 12.1 | | |
| | 0.09563 | 0.0956 | | 12.8 | | |
| 20 | 0.08479 | 0.0848 | 0.0860 ± 0.00138 | 11.3 | 11.5 ± 0.183 | 86.2 |
| | 0.08748 | 0.0875 | | 11.7 | | |
| | 0.08564 | 0.0856 | | 11.4 | | |
| 30 | 0.08319 | 0.0832 | 0.0866 ± 0.00315 | 11.1 | 11.5 ± 0.421 | 86.9 |
| | 0.08721 | 0.0872 | | 11.6 | | |
| | 0.08941 | 0.0894 | | 11.9 | | |

Abbreviations:
SD, standard deviation;
VC, vehicle control (2% Water/1% Acetonitrile)
Note:
For all calculations above, the resulting values are shown with at least three significant figures for display purposes only.

TABLE 17
Quinine Sulfate Effects on CYP2D6 Activity in Pooled Human Liver Microsomes

| Quinine | Dextrorphan formation | | | Specific Activity | | |
|---|---|---|---|---|---|---|
| Sulfate | Raw | Adjusted (μM) | | (pmol/min/mg protein) | | Percent |
| (μM) | (μM) | Individual | Mean ± SD | Individual | Mean ± SD | of VC |
| 0 | 0.04492 | 0.0449 | 0.0476 ± 0.00213 | 12.0 | 12.7 ± 0.568 | 100 |
| (VC) | 0.04963 | 0.0496 | | 13.2 | | |
| | 0.04890 | 0.0489 | | 13.0 | | |
| | 0.04682 | 0.0468 | | 12.5 | | |
| 0.2 | 0.04691 | 0.0469 | 0.0497 ± 0.00255 | 12.5 | 13.3 ± 0.679 | 105 |
| | 0.05186 | 0.0519 | | 13.8 | | |
| | 0.05042 | 0.0504 | | 13.4 | | |
| 2 | 0.04340 | 0.0434 | 0.0428 ± 0.000957 | 11.6 | 11.4 ± 0.255 | 90.0 |
| | 0.04331 | 0.0433 | | 11.5 | | |
| | 0.04170 | 0.0417 | | 11.1 | | |
| 10 | 0.02284 | 0.0228 | 0.0246 ± 0.00194 | 6.09 | 6.57 ± 0.517 | 51.8 |
| | 0.02439 | 0.0244 | | 6.50 | | |
| | 0.02669 | 0.0267 | | 7.12 | | |
| 20 | 0.01777 | 0.0178 | 0.0179 ± 0.000418 | 4.74 | 4.78 ± 0.111 | 37.7 |
| | 0.01840 | 0.0184 | | 4.91 | | |
| | 0.01761 | 0.0176 | | 4.70 | | |
| 30 | 0.01325 | 0.0133 | 0.0130 ± 0.000724 | 3.53 | 3.46 ± 0.193 | 27.3 |
| | 0.01353 | 0.0135 | | 3.61 | | |
| | 0.01216 | 0.0122 | | 3.24 | | |

Abbreviations:
SD, standard deviation;
VC, vehicle control (2% Water/1% Acetonitrile)
Note:
For all calculations above, the resulting values are shown with at least three significant figures for display purposes only.

TABLE 18
Quinine Sulfate Effects on CYP2E1 Activity in Pooled Human Liver Microsomes

| Quinine | 6-Hydroxychlorzoxazone formation | | | Specific Activity | | |
|---|---|---|---|---|---|---|
| Sulfate | Raw | Adjusted (μM) | | (pmol/min/mg protein) | | Percent |
| (μM) | (μM) | Individual | Mean ± SD | Individual | Mean ± SD | of VC |
| 0 | 1.03025 | 1.03 | 1.01 ± 0.0665 | 275 | 269 ± 17.7 | 100 |
| (VC) | 0.94002 | 0.940 | | 251 | | |
| | 0.97509 | 0.975 | | 260 | | |
| | 1.09223 | 1.09 | | 291 | | |
| 0.2 | 1.01368 | 1.01 | 1.03 ± 0.0468 | 270 | 276 ± 12.5 | 102 |
| | 1.00124 | 1.00 | | 267 | | |
| | 1.08783 | 1.09 | | 290 | | |
| 2 | 1.10696 | 1.11 | 1.09 ± 0.0282 | 295 | 290 ± 7.52 | 108 |
| | 1.05499 | 1.05 | | 281 | | |
| | 1.09993 | 1.10 | | 293 | | |
| 10 | 0.94953 | 0.950 | 1.02 ± 0.0841 | 253 | 272 ± 22.4 | 101 |
| | 1.11345 | 1.11 | | 297 | | |
| | 0.99846 | 0.998 | | 266 | | |
| 20 | 1.00415 | 1.00 | 1.05 ± 0.0469 | 268 | 281 ± 12.5 | 104 |
| | 1.05967 | 1.06 | | 283 | | |
| | 1.09737 | 1.10 | | 293 | | |
| 30 | 1.15807 | 1.16 | 1.12 ± 0.0308 | 309 | 300 ± 8.21 | 111 |
| | 1.09771 | 1.10 | | 293 | | |
| | 1.11719 | 1.12 | | 298 | | |

Abbreviations:
SD, standard deviation;
VC, vehicle control (2% Water/1% Acetonitrile)
Note:
For all calculations above, the resulting values are shown with at least three significant figures for display purposes only.

TABLE 19
Quinine Sulfate Effects on CYP3A4 Activity in Pooled Human Liver Microsomes

| Quinine | 6β-Hydroxytestosterone formation | | | Specific Activity | | |
|---|---|---|---|---|---|---|
| Sulfate | Raw | Adjusted (μM) | | (pmol/min/mg protein) | | Percent |
| (μM) | (μM) | Individual | Mean ± SD | Individual | Mean ± SD | of VC |
| 0 | 0.71345 | 0.713 | 0.729 ± 0.0272 | 571 | 583 ± 21.8 | 100 |
| (VC) | 0.69820 | 0.698 | | 559 | | |
| | 0.74975 | 0.750 | | 600 | | |
| | 0.75361 | 0.754 | | 603 | | |
| 0.2 | 0.80554 | 0.806 | 0.807 ± 0.00601 | 644 | 645 ± 4.81 | 111 |
| | 0.80145 | 0.801 | | 641 | | |
| | 0.81328 | 0.813 | | 651 | | |
| 2 | 0.80488 | 0.805 | 0.810 ± 0.00480 | 644 | 648 ± 3.84 | 111 |
| | 0.81068 | 0.811 | | 649 | | |
| | 0.81440 | 0.814 | | 652 | | |
| 10 | 0.75067 | 0.751 | 0.755 ± 0.00627 | 601 | 604 ± 5.02 | 104 |
| | 0.75156 | 0.752 | | 601 | | |
| | 0.76195 | 0.762 | | 610 | | |
| 20 | 0.75257 | 0.753 | 0.771 ± 0.0283 | 602 | 617 ± 22.7 | 106 |
| | 0.80352 | 0.804 | | 643 | | |
| | 0.75661 | 0.757 | | 605 | | |
| 30 | 0.71410 | 0.714 | 0.766 ± 0.0741 | 571 | 613 ± 59.3 | 105 |
| | 0.85083 | 0.851 | | 681 | | |
| | 0.73307 | 0.733 | | 586 | | |

Abbreviations:
SD, standard deviation;
VC, vehicle control (2% Water/1% Acetonitrile)
Note:
For all calculations above, the resulting values are shown with at least three significant figures for display purposes only.

Under these experimental conditions, no tested concentration of quinine sulfate inhibited activity of CYP2E1 (Table 18) or CYP3A4 (Table 19) in human liver microsomes at a statistically significant level (p>0.05 using an unpaired two-tailed t-test).

However, under these experimental conditions, quinine sulfate did inhibit activities of CYP1A2 (Table 11), CYP2A6 (Table 12), CYP2B6 (Table 13), CYP2C8 (Table 14), CYP2C9 (Table 15), CYP2C19 (Table 16), and CYP2D6 (Table 17) in human liver microsomes at one or more of the tested quinine sulfate concentrations at a statistically significant level (p≦0.05 using an unpaired two-tailed t-test).

For CYP2C8 and CYP2D6, IC50 values could be calculated from the inhibition data at these experimental conditions. Quinine sulfate inhibited CYP2C8 activity in human liver microsomes with an IC50 value of 23.7 μM (95% confidence limits: 18.6-30.2 μM) and inhibited CYP2D6 activity in human liver microsomes with an IC50 value of 10.1 μM (95% confidence limits: 8.5-11.9 μM).

### EXAMPLE 3

### Quinine Sulfate Induction/Inhibition of Cytochrome p450 Isozymes

The study of this example was performed to determine if there is induction or inhibition by quinine of cytochrome p450 isozymes CYP1A2, CYP2A6, CYP2B6, CYP2C8, CYP2C9, CYP2C19, CYP2D6, CYP2E1, and CYP3A4. These induction/inhibition studies used freshly isolated human hepatocytes and compared enzymatic activity levels for each of these cytochrome p450 isozymes, using an appropriate enzyme substrate, in the human hepatocytes following in vitro exposure for 48±3 hrs to the presence or absence of quinine sulfate.

Hepatocytes from three human donors were obtained from a cryopreserved hepatocyte bank (In Vitro Technologies, Inc., USA).

Donor 1 was reported to be a 51-year old Caucasian male who died of ischemic stroke, with a medical history including Type 2 diabetes, hypertension, hyperlipidemia, kidney stone removal, sleep apnea, depression and colitis. Serology testing was negative except for cytomegalovirus. Donor 1 was known to smoke tobacco.

Donor 2 was reported to be a 54-year old Caucasian female who died of cardiac arrest, with a medical history including high cholesterol. Serology testing was negative, including cytomegalovirus. Donor 2 was known to smoke tobacco.

Donor 3 was reported to be a 40-year old Caucasian female who died of a drug overdose, with a medical history including hypertension. Serology testing was negative except for cytomegalovirus. Donor 3 had a history of cocaine, opiate and marijuana use, as well as recreational use of libriam, oritab and adovan.

After thawing, viable hepatocytes from each donor were transferred to collagen-coated 48-well plates for attachment in plating medium (DMEM stock (Dulbecco's modified Eagle's medium, supplemented with bovine serum albumin, fructose, N-(2-hydroxyethyl)piperazine-N′-(2-ethanesulfonate) (HEPES), and sodium bicarbonate), supplemented with antibiotics, bovine serum, hydrocortisone, insulin and minimum essential medium (MEM) nonessential amino acids). After attachment to the collagen matrix, plating medium was replaced with sandwich medium (incubation medium supplemented with VITROGEN and incubated until use. All incubations were conducted at 37±1° C., 95% air/5% CO₂ and saturating humidity.

After establishment of the hepatocyte cultures, sandwich medium was removed and the hepatocytes were incubated with incubation solution (DMEM stock supplemented with antibiotics, hydrocortisone, insulin, and MEM non-essential amino acids) containing 5.0, 15, or 30 μM quinine sulfate for 24±1.5 hrs. Incubation solution was aspirated and replaced with incubation solution containing the same concentration of quinine sulfate and incubated for an additional 24±1.5 hrs. After the quinine sulfate treatment period, the incubation solution was replaced with 150 μL Krebs-Henseleit (KHB) buffer supplemented with antibiotics, calcium chloride, heptanoic acid, HEPES, and sodium bicarbonate (supplemented KHB) and incubated for 10 minutes. The supplemented KHB was replaced with 150 μL supplemented KHB containing the appropriate isoform-selective substrate and incubated for 4 hrs prior to termination by adding 150 μL ice-cold methanol, except for the CYP2C8 incubations which were terminated by adding 150 μL acetonitrile. Samples were transferred to cryovials and analyzed after storage at −70° C. Three induction replicates were performed at each quinine sulfate concentration for each cytochrome p450 isozyme.

Analogous vehicle control experiments were also performed to establish a baseline value for enzyme activity in the absence of quinine sulfate. Vehicle control experiments were performed as described above for the test induction incubations, except that the incubation medium included no quinine sulfate. Four replicates were performed of the vehicle control for each donor.

A table of the substrate, substrate concentration, metabolite formed, and metabolite assay method for each CYP isozyme studied is provided below. All substrates were dissolved in acetonitrile as 100× solutions. All 100× substrate solutions were diluted with supplemented KHB to the final concentrations listed below, except for paclitaxel, which was diluted with incubation medium.

TABLE 20
Isoform-selective substrates for cytochrome p450 isozymes in the quinine sulfate
induction/inhibition study.

| | Isoform-selective | Substrate | | | Metabolite |
|---|---|---|---|---|---|
| CYP isoform | substrate | concentration | | Metabolite formed | Assay |
| CYP1A2 | Phenacetin | 100 | μM | acetaminophen | LC/MS |
| CYP2A6 | Coumarin | 100 | μM | 7-hydroxycoumarin, | HPLC-UV |
| | | | | 7-hydroxy coumarin glucuronide, | |
| | | | | 7-hydroxycoumarin sulfate | |
| CYP2B6 | S-Mephenytoin | 1 | mM | nirvanol | LC/MS |
| CYP2C8 | Paclitaxel | 50 | μM | 6-hydroxy paclitaxel | HPLC-UV |
| CYP2C9 | Tolbutamide | 50 | μM | 4′-methylhydroxytolbutamide | LC/MS |
| CYP2C19 | S-Mephenytoin | 100 | μM | 4′-hydroxy mephenytoin | LC/MS |
| CYP2D6 | Dextromethorphan | 16 | μM | dextrorphan | LC/MS |
| CYP2E1 | Chlorzoxazone | 300 | μM | 6-hydroxychlorzoxazone | LC/MS |
| CYP3A4 | Testosterone | 125 | μM | 6β-hydroxy testosterone | HPLC-UV |

Quinine sulfate 50× stock solutions were prepared in water as described above and diluted with incubation medium and acetonitrile to produce incubation solutions with 5.0, 15, and 30 μM quinine sulfate, each containing 2% water and 1% acetonitrile.

Positive controls (n=4) were performed to verify that the test system was sensitive to known inducers by testing induction of CYP1A2 and CYP3A4 by 50 μM omeprazole and 25 μM rifampicin, respectively, using the appropriate isoform-selective enzyme substrate. Following treatment with 50 μM omeprazole, CYP1A2 activity was 1,238%, 521%, and 691% of the vehicle control in human hepatocytes prepared from Donors 1, 2, and 3, respectively. Following treatment with 25 μM rifampin, CYP3A4 activity was >828%, >2,854%, and 1,372% of the VC in human hepatocytes prepared from Donors 1, 2, and 3, respectively. Based on these increasse in activities of CYP1A2 and CYP3A4 following treatment with the known inducers; the hepatocytes from the three donors were considered inducible.

Additionally, reference control samples were included to evaluate inducibility of CYP2B6, CYP2C8, CYP2C9, and CYP2C19 in the test system. The reference controls included 1 mM Phenobarbital (for CYP2B6) or 25 μM rifampicin as the reference inducer. The reference controls showed a statistically significant amount of induction for each hepatocyte donor for CYP2B6, CYP2C8, and CYP2C9, although the amount of induction varied between the three hepatocyte donors for each isozyme. For CYP2C19, rifampin induced CYP2C19 activity in donor 3, but did not induce CYP2C19 activity in donors 1 or 2 at a statistically significant level (p<0.05 using an unpaired two-tailed t-test) although 25 μM rifampin did raise CYP2C19 activity in these donors from undetectable in the vehicle control to levels that were measurable but below the lowest concentration of the standard curve.

Furthermore, interference controls were performed for each CYP isozyme to determine whether or not quinine sulfate or its metabolites interfered with detection of the isoform-specific metabolites. In these controls, performed in duplicate, the hepatocytes were incubated with quinine sulfate as for the test samples, and then incubated with the buffer of the isoform-specific substrate (without substrate) as for the test samples. No interference of quinine sulfate or its metabolite was observed in any of the assays for detection of the isoform-specific metabolites formed in the test systems.

Results for each cytochrome p450 isozyme are shown in Tables 21-29. Significant induction was observed at these experimental conditions for CYP1A2, CYP2A6, CYP2B6, CYP2C9, CYP2E1, and CYP3A4. Additionally, significant inhibition in enzyme activity was observed in all three donors for CYP2D6. Under these experimental conditions, no significant effects on activity of CYP2C19 were observed after exposure to any of the tested concentrations of quinine sulfate. Significance of a change in specific activity from that measured for the vehicle control (0 μM quinine sulfate) was determined using a two-tailed t-test. Mean specific activity values with associated p-values ≦0.05 were deemed to be statistically significant.

TABLE 21
CYP1A2 Activity in Cryopreserved Human Hepatocyte Monolayers Following
48 hr Incubation with Quinine Sulfate Prior to Substrate Addition

| Quinine | Acetaminophen formation | | | Specific Activity | | |
|---|---|---|---|---|---|---|
| Sulfate | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.06486 | 0.0649 | 0.0848 ± 0.0151 | 0.579 | 0.757 ± 0.134 | 100 |
| (VC) | 0.08207 | 0.0821 | | 0.733 | | |
| | 0.09923 | 0.0992 | | 0.886 | | |
| | 0.09301 | 0.0930 | | 0.830 | | |
| 5 | 0.45982 | 0.460 | 0.535 ± 0.0693 | 4.11 | 4.77 ± 0.619 | 631 |
| | 0.54777 | 0.548 | | 4.89 | | |
| | 0.59659 | 0.597 | | 5.33 | | |
| 15 | 1.03551 | 1.04 | 1.18 ± 0.129 | 9.25 | 10.6 ± 1.16 | 1,397 |
| | 1.26374 | 1.26 | | 11.3 | | |
| | 1.25566 | 1.26 | | 11.2 | | |
| 30 | 1.32967 | 1.33 | 1.87 ± 0.472 | 11.9 | 16.7 ± 4.21 | 2,209 |
| | 2.11238 | 2.11 | | 18.9 | | |
| | 2.17695 | 2.18 | | 19.4 | | |

Human Donor 2

| | | | | | | |
|---|---|---|---|---|---|---|
| 0 | 0.77542 | 0.775 | 0.723 ± 0.0364 | 6.92 | 6.46 ± 0.325 | 100 |
| (VC) | 0.71573 | 0.716 | | 6.39 | | |
| | 0.71031 | 0.710 | | 6.34 | | |
| | 0.69119 | 0.691 | | 6.17 | | |
| 5 | 2.14033 | 2.14 | 2.27 ± 0.113 | 19.1 | 20.3 ± 1.01 | 314 |
| | 2.33568 | 2.34 | | 20.9 | | |
| | 2.33768 | 2.34 | | 20.9 | | |
| 15 | 3.31784 | 3.32 | 3.23 ± 0.606 | 29.6 | 28.9 ± 5.41 | 447 |
| | 2.59047 | 2.59 | | 23.1 | | |
| | 3.79339 | 3.79 | | 33.9 | | |
| 30 | 4.42275 | 4.42 | 4.78 ± 0.432 | 39.5 | 42.7 ± 3.85 | 661 |
| | 5.25856 | 5.26 | | 47.0 | | |
| | 4.65354 | 4.65 | | 41.5 | | |

Human Donor 3

| | | | | | | |
|---|---|---|---|---|---|---|
| 0 | 1.31250 | 1.31 | 1.43 ± 0.0809 | 11.7 | 12.8 ± 0.723 | 100 |
| (VC) | 1.48620 | 1.49 | | 13.3 | | |
| | 1.44182 | 1.44 | | 12.9 | | |
| | 1.48042 | 1.48 | | 13.2 | | |
| 5 | 3.50593 | 3.51 | 3.40 ± 0.117 | 31.3 | 30.4 ± 1.05 | 238 |
| | 3.43119 | 3.43 | | 30.6 | | |
| | 3.27616 | 3.28 | | 29.3 | | |
| 15 | 5.16178 | 5.16 | 5.24 ± 0.0977 | 46.1 | 46.8 ± 0.872 | 367 |
| | 5.21633 | 5.22 | | 46.6 | | |
| | 5.35149 | 5.35 | | 47.8 | | |
| 30 | 7.02348 | 7.02 | 7.11 ± 0.104 | 62.7 | 63.5 ± 0.926 | 497 |
| | 7.22674 | 7.23 | | 64.5 | | |
| | 7.08944 | 7.09 | | 63.3 | | |

Abbreviations:
SD, standard deviation;
VC, vehicle control (2% Water/1% Acetonitrile)
Note:
For all calculations above, the resulting values are shown with at least three significant figures for display purposes only.

TABLE 22a
CYP2A6 Activity in Cryopreserved Human Hepatocyte Monolayers Following
48 hr Incubation with Quinine Sulfate Prior to Substrate Addition

| Quinine | Metabolite formation | | | Specific Activity | | |
|---|---|---|---|---|---|---|
| Sulfate | Raw | Adjusted (μM) | | (pmol/min/million cells) | | Percent |
| (μM) | (μM) | Individual | Mean ± SD | Individual | Mean ± SD | of VC |

7-Hydrxoycoumarin (7-HC) Formation: Human Donor 1

| | | | | | | |
|---|---|---|---|---|---|---|
| 0 | 0.00000^{a} | <0.100 | <0.100 ± 0.000 | <0.893 | <0.893 ± 0.000 | 100 |
| (VC) | 0.00000^{a} | <0.100 | | <0.893 | | |
| | 0.00000^{a} | <0.100 | | <0.893 | | |
| | 0.00000^{a} | <0.100 | | <0.893 | | |
| 5 | 0.00000^{a} | <0.100 | <0.100 ± 0.000 | <0.893 | <0.893 ± 0.000 | 100 |
| | 0.00000^{a} | <0.100 | | <0.893 | | |
| | 0.00000^{a} | <0.100 | | <0.893 | | |
| 15 | 0.00000^{a} | <0.100 | <0.100 ± 0.000 | <0.893 | <0.893 ± 0.000 | 100 |
| | 0.00000^{a} | <0.100 | | <0.893 | | |
| | 0.00000^{a} | <0.100 | | <0.893 | | |
| 30 | 0.00000^{a} | <0.100 | <0.100 ± 0.000 | <0.893 | <0.893 ± 0.000 | 100 |
| | 0.00000^{a} | <0.100 | | <0.893 | | |
| | 0.00000^{a} | <0.100 | | <0.893 | | |

7-Hydrxoycoumarin (7-HC) Formation: Human Donor 2

| | | | | | | |
|---|---|---|---|---|---|---|
| 0 | 0.03221^{a} | <0.100 | <0.100 ± 0.000 | <0.893 | <0.893 ± 0.000 | 100 |
| (VC) | 0.02788^{a} | <0.100 | | <0.893 | | |
| | 0.03128^{a} | <0.100 | | <0.893 | | |
| | 0.02760^{a} | <0.100 | | <0.893 | | |
| 5 | 0.04062^{a} | <0.100 | <0.100 ± 0.000 | <0.893 | <0.893 ± 0.000 | 100 |
| | 0.04125^{a} | <0.100 | | <0.893 | | |
| | 0.03795^{a} | <0.100 | | <0.893 | | |
| 15 | 0.04415^{a} | <0.100 | <0.100 ± 0.000 | <0.893 | <0.893 ± 0.000 | 100 |
| | 0.04821^{a} | <0.100 | | <0.893 | | |
| | 0.04713^{a} | <0.100 | | <0.893 | | |
| 30 | 0.04598^{a} | <0.100 | <0.100 ± 0.000 | <0.893 | <0.893 ± 0.000 | 100 |
| | 0.04748^{a} | <0.100 | | <0.893 | | |
| | 0.04630^{a} | <0.100 | | <0.893 | | |

7-Hydrxoycoumarin (7-HC) Formation: Human Donor 3

| | | | | | | |
|---|---|---|---|---|---|---|
| 0 | 0.19144 | 0.191 | 0.192 ± 0.0269 | 1.71 | 1.72 ± 0.240 | 100 |
| (VC) | 0.22555 | 0.226 | | 2.01 | | |
| | 0.19183 | 0.192 | | 1.71 | | |
| | 0.15974 | 0.160 | | 1.43 | | |
| 5 | 0.26361 | 0.264 | 0.229 ± 0.0360 | 2.35 | 2.04 ± 0.321 | 119 |
| | 0.23122 | 0.231 | | 2.06 | | |
| | 0.19174 | 0.192 | | 1.71 | | |
| 15 | 0.21158 | 0.212 | 0.202 ± 0.0335 | 1.89 | 1.81 ± 0.299 | 105 |
| | 0.23022 | 0.230 | | 2.06 | | |
| | 0.16515 | 0.165 | | 1.47 | | |
| 30 | 0.14596 | 0.146 | 0.142 ± 0.00451 | 1.30 | 1.27 ± 0.0402 | 74.1 |
| | 0.14387 | 0.144 | | 1.28 | | |
| | 0.13732 | 0.137 | | 1.23 | | |

Abbreviations:
SD, standard deviation;
VC, vehicle control (2% Water/1% Acetonitrile)
^{a}The observed analyzed value (μM) was below the lowest concentration on the standard curve (0.1 μM).
Note:
For all calculations above, the resulting values are shown with at least three significant figures for display purposes only.

TABLE 22b
CYP2A6 Activity in Cryopreserved Human Hepatocyte Monolayers Following
48 hr Incubation with Quinine Sulfate Prior to Substrate Addition

| Quinine | Metabolite formation | | | Specific Activity | | |
|---|---|---|---|---|---|---|
| Sulfate | Raw | Adjusted (μM) | | (pmol/min/million cells) | | Percent |
| (μM) | (μM) | Individual | Mean ± SD | Individual | Mean ± SD | of VC |

7-Hydroxycoumarin Glucuronide (7-HCG) Formation: Human Donor 1

| | | | | | | |
|---|---|---|---|---|---|---|
| 0 | 0.02935^{b} | <0.0500 | <0.0500 ± 0.000 | <0.446 | <0.446 ± 0.000 | 100 |
| (VC) | 0.02927^{b} | <0.0500 | | <0.446 | | |
| | 0.02000^{b} | <0.0500 | | <0.446 | | |
| | 0.00000^{b} | <0.0500 | | <0.446 | | |
| 5 | 0.06356 | 0.0636 | 0.0583 ± 0.00637 | 0.568 | 0.520 ± 0.0569 | >117 |
| | 0.06000 | 0.0600 | | 0.536 | | |
| | 0.05119 | 0.0512 | | 0.457 | | |
| 15 | 0.08491 | 0.0849 | 0.0828 ± 0.00206 | 0.758 | 0.739 ± 0.0184 | >166 |
| | 0.08273 | 0.0827 | | 0.739 | | |
| | 0.08080 | 0.0808 | | 0.721 | | |
| 30 | 0.05843 | 0.0584 | 0.0552 ± 0.00383 | 0.522 | 0.493 ± 0.0342 | >110 |
| | 0.05631 | 0.0563 | | 0.503 | | |
| | 0.05099 | 0.0510 | | 0.455 | | |

7-Hydroxycoumarin Glucuronide (7-HCG) Formation: Human Donor 2

| | | | | | | |
|---|---|---|---|---|---|---|
| 0 | 0.66626 | 0.666 | 0.676 ± 0.0525 | 5.95 | 6.03 ± 0.469 | 100 |
| (VC) | 0.64824 | 0.648 | | 5.79 | | |
| | 0.75216 | 0.752 | | 6.72 | | |
| | 0.63604 | 0.636 | | 5.68 | | |
| 5 | 0.89822 | 0.898 | 0.983 ± 0.0932 | 8.02 | 8.77 ± 0.832 | 145 |
| | 0.96682 | 0.967 | | 8.63 | | |
| | 1.08264 | 1.08 | | 9.67 | | |
| 15 | 1.04287 | 1.04 | 1.15 ± 0.0941 | 9.31 | 10.3 ± 0.841 | 170 |
| | 1.21285 | 1.21 | | 10.8 | | |
| | 1.19798 | 1.20 | | 10.7 | | |
| 30 | 0.79053 | 0.791 | 0.833 ± 0.0509 | 7.06 | 7.44 ± 0.454 | 123 |
| | 0.81869 | 0.819 | | 7.31 | | |
| | 0.88926 | 0.889 | | 7.94 | | |

7-Hydroxycoumarin Glucuronide (7-HCG) Formation: Human Donor 3

| | | | | | | |
|---|---|---|---|---|---|---|
| 0 | 11.76824 | 11.8 | 11.4 ± 0.670 | 105 | 102 ± 5.98 | 100 |
| (VC) | 11.47721 | 11.5 | | 102 | | |
| | 11.84171 | 11.8 | | 106 | | |
| | 10.39355 | 10.4 | | 92.8 | | |
| 5 | 14.50267 | 14.5 | 14.5 ± 0.194 | 129 | 130 ± 1.74 | 128 |
| | 14.74802 | 14.7 | | 132 | | |
| | 14.36402 | 14.4 | | 128 | | |
| 15 | 13.35789 | 13.4 | 12.4 ± 1.31 | 119 | 111 ± 11.7 | 109 |
| | 12.98746 | 13.0 | | 116 | | |
| | 10.93199 | 10.9 | | 97.6 | | |
| 30 | 8.99318 | 8.99 | 8.80 ± 0.263 | 80.3 | 78.5 ± 2.35 | 77.4 |
| | 8.89833 | 8.90 | | 79.4 | | |
| | 8.49818 | 8.50 | | 75.9 | | |

Abbreviations:
SD, standard deviation;
VC, vehicle control (2% Water/1% Acetonitrile)
^{b}The observed analyzed value (μM) was below the lowest concentration on the standard curve (0.05 μM).
Note:
For all calculations above, the resulting values are shown with at least three significant figures for display purposes only.

TABLE 22c
CYP2A6 Activity in Cryopreserved Human Hepatocyte Monolayers Following
48 hr Incubation with Quinine Sulfate Prior to Substrate Addition

| Quinine | Metabolite formation | | | Specific Activity | | |
|---|---|---|---|---|---|---|
| Sulfate | Raw | Adjusted (μM) | | (pmol/min/million cells) | | Percent |
| (μM) | (μM) | Individual | Mean ± SD | Individual | Mean ± SD | of VC |

7-Hydrxoycoumarin Sulfate (7-HCS) Formation: Human Donor 1

| | | | | | | |
|---|---|---|---|---|---|---|
| 0 | 0.00000^{c} | <0.150 | <0.150 ± 0.000 | <1.34 | <1.34 ± 0.000 | 100 |
| (VC) | 0.00000^{c} | <0.150 | | <1.34 | | |
| | 0.00000^{c} | <0.150 | | <1.34 | | |
| | 0.00000^{c} | <0.150 | | <1.34 | | |
| 5 | 0.00000^{c} | <0.150 | <0.150 ± 0.000 | <1.34 | <1.34 ± 0.000 | 100 |
| | 0.00000^{c} | <0.150 | | <1.34 | | |
| | 0.00000^{c} | <0.150 | | <1.34 | | |
| 15 | 0.03775^{c} | <0.150 | <0.150 ± 0.000 | <1.34 | <1.34 ± 0.000 | 100 |
| | 0.00000^{c} | <0.150 | | <1.34 | | |
| | 0.00000^{c} | <0.150 | | <1.34 | | |
| 30 | 0.00000^{c} | <0.150 | <0.150 ± 0.000 | <1.34 | <1.34 ± 0.000 | 100 |
| | 0.00000^{c} | <0.150 | | <1.34 | | |
| | 0.00000^{c} | <0.150 | | <1.34 | | |

7-Hydrxoycoumarin Sulfate (7-HCS) Formation: Human Donor 2

| | | | | | | |
|---|---|---|---|---|---|---|
| 0 | 0.15599 | 0.156 | <0.160 ± 0.0131 | 1.39 | <1.43 ± 0.117 | 100 |
| (VC) | 0.15567 | 0.156 | | 1.39 | | |
| | 0.17960 | 0.180 | | 1.60 | | |
| | 0.14500^{c} | <0.150 | | <1.34 | | |
| 5 | 0.19160 | 0.192 | 0.206 ± 0.0166 | 1.71 | 1.84 ± 0.149 | >128 |
| | 0.20138 | 0.201 | | 1.80 | | |
| | 0.22404 | 0.224 | | 2.00 | | |
| 15 | 0.20786 | 0.208 | 0.232 ± 0.0207 | 1.86 | 2.07 ± 0.184 | >145 |
| | 0.24449 | 0.244 | | 2.18 | | |
| | 0.24270 | 0.243 | | 2.17 | | |
| 30 | 0.15872 | 0.159 | 0.171 ± 0.0142 | 1.42 | 1.53 ± 0.127 | >107 |
| | 0.16749 | 0.167 | | 1.50 | | |
| | 0.18650 | 0.187 | | 1.67 | | |

7-Hydrxoycoumarin Sulfate (7-HCS) Formation: Human Donor 3

| | | | | | | |
|---|---|---|---|---|---|---|
| 0 | 0.63051 | 0.631 | 0.608 ± 0.0362 | 5.63 | 5.43 ± 0.323 | 100 |
| (VC) | 0.61143 | 0.611 | | 5.46 | | |
| | 0.63514 | 0.635 | | 5.67 | | |
| | 0.55636 | 0.556 | | 4.97 | | |
| 5 | 0.62226 | 0.622 | 0.645 ± 0.0202 | 5.56 | 5.76 ± 0.181 | 106 |
| | 0.65964 | 0.660 | | 5.89 | | |
| | 0.65431 | 0.654 | | 5.84 | | |
| 15 | 0.55588 | 0.556 | 0.533 ± 0.0269 | 4.96 | 4.76 ± 0.240 | 87.6 |
| | 0.54004 | 0.540 | | 4.82 | | |
| | 0.50338 | 0.503 | | 4.49 | | |
| 30 | 0.32426 | 0.324 | 0.333 ± 0.0171 | 2.90 | 2.98 ± 0.153 | 54.8 |
| | 0.35297 | 0.353 | | 3.15 | | |
| | 0.32253 | 0.323 | | 2.88 | | |

Abbreviations:
SD, standard deviation;
VC, vehicle control (2% Water/1% Acetonitrile)
^{c}The observed analyzed value (μM) was below the lowest concentration on the standard curve (0.15 μM).
Note:
For all calculations above, the resulting values are shown with at least three significant figures for display purposes only.

TABLE 22d
CYP2A6 Activity in Cryopreserved Human Hepatocyte Monolayers Following
48 hr Incubation with Quinine Sulfate Prior to Substrate Addition

| Quinine | Metabolite formation | | | Specific Activity | | |
|---|---|---|---|---|---|---|
| Sulfate | Raw | Adjusted (μM) | | (pmol/min/million cells) | | Percent |
| (μM) | (μM) | Individual | Mean ± SD | Individual | Mean ± SD | of VC |

Total Metabolite Formation: Human Donor 1

| | | | | | | |
|---|---|---|---|---|---|---|
| 0 | 0.0294^{d} | 0.300 | 0.300 ± 0.000 | 2.68 | 2.68 ± 0.000 | 100 |
| (VC) | 0.0293^{d} | 0.300 | | 2.68 | | |
| | 0.0200^{d} | 0.300 | | 2.68 | | |
| | 0.000^{d} | 0.300 | | 2.68 | | |
| 5 | 0.0636^{e} | 0.314 | 0.308 ± 0.00637 | 2.80 | 2.75 ± 0.0569 | 103 |
| | 0.0600^{e} | 0.310 | | 2.77 | | |
| | 0.0512^{e} | 0.301 | | 2.69 | | |
| 15 | 0.123^{e} | 0.335 | 0.333 ± 0.00206 | 2.99 | 2.97 ± 0.0184 | 111 |
| | 0.0827^{e} | 0.333 | | 2.97 | | |
| | 0.0808^{e} | 0.331 | | 2.95 | | |
| 30 | 0.0584^{e} | 0.308 | 0.305 ± 0.00383 | 2.75 | 2.73 ± 0.0342 | 102 |
| | 0.0563^{e} | 0.306 | | 2.73 | | |
| | 0.0510^{e} | 0.301 | | 2.69 | | |

Total Metabolite Formation: Human Donor 2

| | | | | | | |
|---|---|---|---|---|---|---|
| 0 | 0.854^{f} | <0.922 | <0.936 ± 0.0655 | <8.23 | <8.36 ± 0.585 | 100 |
| (VC) | 0.832^{f} | <0.904 | | <8.07 | | |
| | 0.963^{f} | <1.03 | | <9.21 | | |
| | 0.809^{e} | <0.886 | | <7.91 | | |
| 5 | 1.13^{f} | <1.19 | <1.29 ± 0.110 | <10.6 | <11.5 ± 0.980 | 138 |
| | 1.21^{f} | <1.27 | | <11.3 | | |
| | 1.34^{f} | <1.41 | | <12.6 | | |
| 15 | 1.29^{f} | <1.35 | <1.48 ± 0.115 | <12.1 | <13.2 ± 1.02 | 158 |
| | 1.51^{f} | <1.56 | | <13.9 | | |
| | 1.49^{f} | <1.54 | | <13.8 | | |
| 30 | 0.995^{f} | <1.05 | <1.10 ± 0.0651 | <9.37 | <9.85 ± 0.581 | 118 |
| | 1.03^{f} | <1.09 | | <9.70 | | |
| | 1.12^{f} | <1.18 | | <10.5 | | |

Total Metabolite Formation: Human Donor 3

| | | | | | | |
|---|---|---|---|---|---|---|
| 0 | 12.6 | 12.6 | 12.2 ± 0.724 | 112 | 109 ± 6.46 | 100 |
| (VC) | 12.3 | 12.3 | | 110 | | |
| | 12.7 | 12.7 | | 113 | | |
| | 11.1 | 11.1 | | 99.2 | | |
| 5 | 15.4 | 15.4 | 15.4 ± 0.215 | 137 | 138 ± 1.92 | 127 |
| | 15.6 | 15.6 | | 140 | | |
| | 15.2 | 15.2 | | 136 | | |
| 15 | 14.1 | 14.1 | 13.2 ± 1.36 | 126 | 118 ± 12.2 | 108 |
| | 13.8 | 13.8 | | 123 | | |
| | 11.6 | 11.6 | | 104 | | |
| 30 | 9.46 | 9.46 | 9.27 ± 0.274 | 84.5 | 82.8 ± 2.45 | 76.2 |
| | 9.40 | 9.40 | | 83.9 | | |
| | 8.96 | 8.96 | | 80.0 | | |

Abbreviations:
SD, standard deviation;
VC, vehicle control (2% Water/1% Acetonitrile
^{d}The observed analyzed value (μM) for all metabolites were below the lowest concentration on the corresponding standard curve.
^{e}The observed analyzed value (μM) for 7-HC & 7-7-HCS metabolites were below the lowest concentration on the corresponding standard curve.
^{f}The observed analyzed value (μM) for 7-HC metabolite was below the lowest concentration on the corresponding standard curve.
Note:
For all calculations above, the resulting values are shown with at least three significant figures for display purposes only.

TABLE 23
CYP2B6 Activity in Cryopreserved Human Hepatocyte Monolayers Following
48 hr Incubation with Quinine Sulfate Prior to Substrate Addition

| Quinine | Nirvanol formation | | | Specific Activity | | |
|---|---|---|---|---|---|---|
| Sulfate | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.02197^{a} | <0.0250 | <0.0250 ± 0.000 | <0.223 | <0.223 ± 0.000 | 100 |
| (VC) | 0.02305^{a} | <0.0250 | | <0.223 | | |
| | 0.02206^{a} | <0.0250 | | <0.223 | | |
| | 0.02317^{a} | <0.0250 | | <0.223 | | |
| 5 | 0.02156^{a} | <0.0250 | <0.0251 ± 0.000150 | <0.223 | <0.224 ± 0.00134 | 100 |
| | 0.02248^{a} | <0.0250 | | <0.223 | | |
| | 0.02526 | 0.0253 | | 0.226 | | |
| 15 | 0.02705 | 0.0271 | <0.0257 ± 0.00118 | 0.242 | <0.229 ± 0.0106 | 103 |
| | 0.02400^{a} | <0.0250 | | <0.223 | | |
| | 0.02463^{a} | <0.0250 | | <0.223 | | |
| 30 | 0.02523 | 0.0252 | <0.0251 ± 0.000133 | 0.225 | <0.224 ± 0.00119 | 100 |
| | 0.02301^{a} | <0.0250 | | <0.223 | | |
| | 0.02499^{a} | <0.0250 | | <0.223 | | |

Human Donor 2

| | | | | | | |
|---|---|---|---|---|---|---|
| 0 | 0.09455 | 0.0946 | 0.0941 ± 0.00579 | 0.844 | 0.840 ± 0.0517 | 100 |
| (VC) | 0.08720 | 0.0872 | | 0.779 | | |
| | 0.09344 | 0.0934 | | 0.834 | | |
| | 0.10134 | 0.101 | | 0.905 | | |
| 5 | 0.12757 | 0.128 | 0.133 ± 0.0107 | 1.14 | 1.19 ± 0.0952 | 141 |
| | 0.12634 | 0.126 | | 1.13 | | |
| | 0.14539 | 0.145 | | 1.30 | | |
| 15 | 0.23252 | 0.233 | 0.169 ± 0.0554 | 2.08 | 1.51 ± 0.494 | 179 |
| | 0.13454 | 0.135 | | 1.20 | | |
| | 0.13886 | 0.139 | | 1.24 | | |
| 30 | 0.09168 | 0.0917 | 0.0883 ± 0.00387 | 0.819 | 0.788 ± 0.0346 | 93.8 |
| | 0.08913 | 0.0891 | | 0.796 | | |
| | 0.08407 | 0.0841 | | 0.751 | | |

Human Donor 3

| | | | | | | |
|---|---|---|---|---|---|---|
| 0 | 0.46532 | 0.465 | 0.482 ± 0.0118 | 4.15 | 4.31 ± 0.105 | 100 |
| (VC) | 0.49049 | 0.490 | | 4.38 | | |
| | 0.48994 | 0.490 | | 4.37 | | |
| | 0.48306 | 0.483 | | 4.31 | | |
| 5 | 0.69803 | 0.698 | 0.695 ± 0.00644 | 6.23 | 6.20 ± 0.0575 | 144 |
| | 0.68735 | 0.687 | | 6.14 | | |
| | 0.69894 | 0.699 | | 6.24 | | |
| 15 | 0.67487 | 0.675 | 0.688 ± 0.0130 | 6.03 | 6.14 ± 0.116 | 143 |
| | 0.68813 | 0.688 | | 6.14 | | |
| | 0.70089 | 0.701 | | 6.26 | | |
| 30 | 0.53868 | 0.539 | 0.542 ± 0.00692 | 4.81 | 4.84 ± 0.0618 | 112 |
| | 0.53780 | 0.538 | | 4.80 | | |
| | 0.55020 | 0.550 | | 4.91 | | |

Abbreviations:
SD, standard deviation;
VC, vehicle control (2% Water/1% Acetonitrile)
^{a}The observed analyzed value (μM) was below the lowest concentration on the standard curve (0.025 μM).
Note:
For all calculations above, the resulting values are shown with at least three significant figures for display purposes only.

TABLE 24
CYP2C8 Activity in Cryopreserved Human Hepatocyte Monolayers Following
48 hr Incubation with Quinine Sulfate Prior to Substrate Addition

| Quinine | 6-Hydroxypaclitaxel formation | | | Specific Activity | | |
|---|---|---|---|---|---|---|
| Sulfate | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.04814^{a} | <0.0500 | <0.0503 ± 0.000575 | <0.446 | <0.449 ± 0.00513 | 100 |
| (VC) | 0.05115 | 0.0512 | | 0.457 | | |
| | 0.03215^{a} | <0.0500 | | <0.446 | | |
| | 0.03117^{a} | <0.0500 | | <0.446 | | |
| 5 | 0.06105 | 0.0611 | <0.0555 ± 0.00553 | 0.545 | <0.496 ± 0.0493 | 110 |
| | 0.05551 | 0.0555 | | 0.496 | | |
| | 0.04364^{a} | <0.0500 | | <0.446 | | |
| 15 | 0.04752^{a} | <0.0500 | <0.0520 ± 0.00190 | <0.446 | <0.465 ± 0.0170 | 103 |
| | 0.05376 | 0.0538 | | 0.480 | | |
| | 0.05238 | 0.0524 | | 0.468 | | |
| 30 | 0.10109 | 0.101 | 0.0741 ± 0.0240 | 0.903 | 0.661 ± 0.214 | >147 |
| | 0.06583 | 0.0658 | | 0.588 | | |
| | 0.05528 | 0.0553 | | 0.494 | | |

Human Donor 2

| | | | | | | |
|---|---|---|---|---|---|---|
| 0 | 0.12531 | 0.125 | 0.115 ± 0.0113 | 1.12 | 1.02 ± 0.101 | 100 |
| (VC) | 0.12174 | 0.122 | | 1.09 | | |
| | 0.11180 | 0.112 | | 0.998 | | |
| | 0.10014 | 0.100 | | 0.894 | | |
| 5 | 0.13226 | 0.132 | 0.138 ± 0.00531 | 1.18 | 1.23 ± 0.0474 | 120 |
| | 0.14278 | 0.143 | | 1.27 | | |
| | 0.13872 | 0.139 | | 1.24 | | |
| 15 | 0.10405 | 0.104 | 0.0990 ± 0.00439 | 0.929 | 0.884 ± 0.0392 | 86.3 |
| | 0.09618 | 0.0962 | | 0.859 | | |
| | 0.09675 | 0.0968 | | 0.864 | | |
| 30 | 0.11207 | 0.112 | 0.101 ± 0.0142 | 1.00 | 0.902 ± 0.127 | 88.0 |
| | 0.10604 | 0.106 | | 0.947 | | |
| | 0.08498 | 0.0850 | | 0.759 | | |

Human Donor 3

| | | | | | | |
|---|---|---|---|---|---|---|
| 0 | 0.69565 | 0.696 | 0.639 ± 0.0405 | 6.21 | 5.71 ± 0.362 | 100 |
| (VC) | 0.63615 | 0.636 | | 5.68 | | |
| | 0.62439 | 0.624 | | 5.57 | | |
| | 0.60039 | 0.600 | | 5.36 | | |
| 5 | 0.81597 | 0.816 | 0.770 ± 0.0471 | 7.29 | 6.87 ± 0.420 | 120 |
| | 0.77136 | 0.771 | | 6.89 | | |
| | 0.72185 | 0.722 | | 6.45 | | |
| 15 | 0.75114 | 0.751 | 0.688 ± 0.0546 | 6.71 | 6.15 ± 0.487 | 108 |
| | 0.65993 | 0.660 | | 5.89 | | |
| | 0.65366 | 0.654 | | 5.84 | | |
| 30 | 0.56094 | 0.561 | 0.520 ± 0.0609 | 5.01 | 4.64 ± 0.543 | 81.3 |
| | 0.44989 | 0.450 | | 4.02 | | |
| | 0.54860 | 0.549 | | 4.90 | | |

Abbreviations:
SD, standard deviation;
VC, vehicle control (2% Water/1% Acetonitrile)
^{a}The observed analyzed value (μM) was below the lowest concentration on the standard curve (0.05 μM).
Note:
For all calculations above, the resulting values are shown with at least three significant figures for display purposes only.

TABLE 25
CYP2C9 Activity in Cryopreserved Human Hepatocyte Monolayers Following
48 hr Incubation with Quinine Sulfate Prior to Substrate Addition

| Quinine | 4′-Methylhydroxytolbutamide formation | | | Specific Activity | | |
|---|---|---|---|---|---|---|
| Sulfate | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.00946^{a} | <0.0100 | <0.0107 ± 0.000943 | <0.0893 | <0.0955 ± 0.00842 | 100 |
| (VC) | 0.01202 | 0.0120 | | 0.107 | | |
| | 0.01074 | 0.0107 | | 0.0959 | | |
| | 0.01004 | 0.0100 | | 0.0896 | | |
| 5 | 0.00910^{a} | <0.0100 | <0.0130 ± 0.00266 | <0.0893 | <0.116 ± 0.0237 | 121 |
| | 0.01382 | 0.0138 | | 0.123 | | |
| | 0.01511 | 0.0151 | | 0.135 | | |
| 15 | 0.01585 | 0.0159 | 0.0188 ± 0.00310 | 0.142 | 0.168 ± 0.0276 | >176 |
| | 0.01860 | 0.0186 | | 0.166 | | |
| | 0.02203 | 0.0220 | | 0.197 | | |
| 30 | 0.01439 | 0.0144 | 0.0183 ± 0.00498 | 0.128 | 0.163 ± 0.0445 | >171 |
| | 0.01649 | 0.0165 | | 0.147 | | |
| | 0.02387 | 0.0239 | | 0.213 | | |

Human Donor 2

| | | | | | | |
|---|---|---|---|---|---|---|
| 0 | 0.10405 | 0.104 | 0.107 ± 0.00398 | 0.929 | 0.960 ± 0.0355 | 100 |
| (VC) | 0.11024 | 0.110 | | 0.984 | | |
| | 0.10412 | 0.104 | | 0.930 | | |
| | 0.11158 | 0.112 | | 0.996 | | |
| 5 | 0.14800 | 0.148 | 0.148 ± 0.0106 | 1.32 | 1.32 ± 0.0949 | 138 |
| | 0.15853 | 0.159 | | 1.42 | | |
| | 0.13728 | 0.137 | | 1.23 | | |
| 15 | 0.15402 | 0.154 | 0.151 ± 0.00718 | 1.38 | 1.35 ± 0.0641 | 140 |
| | 0.14266 | 0.143 | | 1.27 | | |
| | 0.15595 | 0.156 | | 1.39 | | |
| 30 | 0.14602 | 0.146 | 0.135 ± 0.0185 | 1.30 | 1.20 ± 0.165 | 125 |
| | 0.14451 | 0.145 | | 1.29 | | |
| | 0.11326 | 0.113 | | 1.01 | | |

Human Donor 3

| | | | | | | |
|---|---|---|---|---|---|---|
| 0 | 1.37089 | 1.37 | 1.39 ± 0.0314 | 12.2 | 12.4 ± 0.280 | 100 |
| (VC) | 1.36476 | 1.36 | | 12.2 | | |
| | 1.41110 | 1.41 | | 12.6 | | |
| | 1.42963 | 1.43 | | 12.8 | | |
| 5 | 1.69335 | 1.69 | 1.76 ± 0.0624 | 15.1 | 15.7 ± 0.557 | 126 |
| | 1.75814 | 1.76 | | 15.7 | | |
| | 1.81810 | 1.82 | | 16.2 | | |
| 15 | 1.78915 | 1.79 | 1.86 ± 0.0644 | 16.0 | 16.6 ± 0.575 | 133 |
| | 1.91373 | 1.91 | | 17.1 | | |
| | 1.87950 | 1.88 | | 16.8 | | |
| 30 | 1.44442 | 1.44 | 1.48 ± 0.0370 | 12.9 | 13.2 ± 0.330 | 106 |
| | 1.47529 | 1.48 | | 13.2 | | |
| | 1.51802 | 1.52 | | 13.6 | | |

Abbreviations:
SD, standard deviation;
VC, vehicle control (2% Water/1% Acetonitrile)
^{a}The observed analyzed value (μM) was below the lowest concentration on the standard curve (0.01 μM).
Note:
For all calculations above, the resulting values are shown with at least three significant figures for display purposes only.

TABLE 26
CYP2C19 Activity in Cryopreserved Human Hepatocyte Monolayers Following
48 hr Incubation with Quinine Sulfate Prior to Substrate Addition

| Quinine | 4′-Hydroxymephenytoin formation | | | Specific Activity | | |
|---|---|---|---|---|---|---|
| Sulfate | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.00000^{a} | <0.0500 | <0.0500 ± 0.000 | <0.446 | <0.446 ± 0.000 | 100 |
| (VC) | 0.00000^{a} | <0.0500 | | <0.446 | | |
| | 0.00000^{a} | <0.0500 | | <0.446 | | |
| | 0.00000^{a} | <0.0500 | | <0.446 | | |
| 5 | 0.00000^{a} | <0.0500 | <0.0500 ± 0.000 | <0.446 | <0.446 ± 0.000 | 100 |
| | 0.00000^{a} | <0.0500 | | <0.446 | | |
| | 0.00000^{a} | <0.0500 | | <0.446 | | |
| 15 | 0.00000^{a} | <0.0500 | <0.0500 ± 0.000 | <0.446 | <0.446 ± 0.000 | 100 |
| | 0.00000^{a} | <0.0500 | | <0.446 | | |
| | 0.00000^{a} | <0.0500 | | <0.446 | | |
| 30 | 0.00000^{a} | <0.0500 | <0.0500 ± 0.000 | <0.446 | <0.446 ± 0.000 | 100 |
| | 0.00000^{a} | <0.0500 | | <0.446 | | |
| | 0.00000^{a} | <0.0500 | | <0.446 | | |

Human Donor 2

| | | | | | | |
|---|---|---|---|---|---|---|
| 0 | 0.00000^{a} | <0.0500 | <0.0500 ± 0.000 | <0.446 | <0.446 ± 0.000 | 100 |
| (VC) | 0.00000^{a} | <0.0500 | | <0.446 | | |
| | 0.00000^{a} | <0.0500 | | <0.446 | | |
| | 0.00000^{a} | <0.0500 | | <0.446 | | |
| 5 | 0.00000^{a} | <0.0500 | <0.0500 ± 0.000 | <0.446 | <0.446 ± 0.000 | 100 |
| | 0.00000^{a} | <0.0500 | | <0.446 | | |
| | 0.00000^{a} | <0.0500 | | <0.446 | | |
| 15 | 0.00000^{a} | <0.0500 | <0.0500 ± 0.000 | <0.446 | <0.446 ± 0.000 | 100 |
| | 0.00000^{a} | <0.0500 | | <0.446 | | |
| | 0.00000^{a} | <0.0500 | | <0.446 | | |
| 30 | 0.00000^{a} | <0.0500 | <0.0500 ± 0.000 | <0.446 | <0.446 ± 0.000 | 100 |
| | 0.00000^{a} | <0.0500 | | <0.446 | | |
| | 0.00000^{a} | <0.0500 | | <0.446 | | |

Human Donor 3

| | | | | | | |
|---|---|---|---|---|---|---|
| 0 | 0.37125 | 0.371 | 0.400 ± 0.0245 | 3.31 | 3.58 ± 0.219 | 100 |
| (VC) | 0.39343 | 0.393 | | 3.51 | | |
| | 0.40738 | 0.407 | | 3.64 | | |
| | 0.42964 | 0.430 | | 3.84 | | |
| 5 | 0.50097 | 0.501 | 0.506 ± 0.0500 | 4.47 | 4.51 ± 0.447 | 126 |
| | 0.45790 | 0.458 | | 4.09 | | |
| | 0.55766 | 0.558 | | 4.98 | | |
| 15 | 0.51345 | 0.513 | 0.509 ± 0.0218 | 4.58 | 4.54 ± 0.195 | 127 |
| | 0.48475 | 0.485 | | 4.33 | | |
| | 0.52763 | 0.528 | | 4.71 | | |
| 30 | 0.43428 | 0.434 | 0.453 ± 0.0167 | 3.88 | 4.05 ± 0.149 | 113 |
| | 0.46210 | 0.462 | | 4.13 | | |
| | 0.46407 | 0.464 | | 4.14 | | |

Abbreviations:
SD, standard deviation;
VC, vehicle control (2% Water/1% Acetonitrile)
^{a}The observed analyzed value (μM) was below the lowest concentration on the standard curve (0.05 μM).
Note:
For all calculations above, the resulting values are shown with at least three significant figures for display purposes only.

TABLE 27
CYP2D6 Activity in Cryopreserved Human Hepatocyte Monolayers Following
48 hr Incubation with Quinine Sulfate Prior to Substrate Addition

| Quinine | Dextrorphan formation | | | specific Activity | | |
|---|---|---|---|---|---|---|
| Sulfate | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.00470^{a} | <0.0100 | <0.0100 ± 0.000 | <0.0893 | <0.0893 ± 0.000 | 100 |
| (VC) | 0.00482^{a} | <0.0100 | (0.004855) | <0.0893 | | |
| | 0.00478^{a} | <0.0100 | | <0.0893 | | |
| | 0.00512^{a} | <0.0100 | | <0.0893 | | |
| 5 | 0.00343^{a} | <0.0100 | <0.0100 ± 0.000 | <0.0893 | <0.0893 ± 0.000 | 100 |
| | 0.00397^{a} | <0.0100 | (0.003703) | <0.0893 | | (76.3) |
| | 0.00371^{a} | <0.0100 | | <0.0893 | | |
| 15 | 0.00413^{a} | <0.0100 | <0.0100 ± 0.000 | <0.0893 | <0.0893 ± 0.000 | 100 |
| | 0.00413^{a} | <0.0100 | (0.004280) | <0.0893 | | (88.2) |
| | 0.00458^{a} | <0.0100 | | <0.0893 | | |
| 30 | 0.00366^{a} | <0.0100 | <0.0100 ± 0.000 | <0.0893 | <0.0893 ± 0.000 | 100 |
| | 0.00412^{a} | <0.0100 | (0.003810) | <0.0893 | | (78.5) |
| | 0.00381^{a} | <0.0100 | | <0.0893 | | |

Human Donor 2

| | | | | | | |
|---|---|---|---|---|---|---|
| 0 | 0.14613 | 0.146 | 0.149 ± 0.00379 | 1.30 | 1.33 ± 0.0338 | 100 |
| (VC) | 0.14582 | 0.146 | | 1.30 | | |
| | 0.15400 | 0.154 | | 1.38 | | |
| | 0.14881 | 0.149 | | 1.33 | | |
| 5 | 0.05584 | 0.0558 | 0.0540 ± 0.00206 | 0.499 | 0.482 ± 0.0184 | 36.3 |
| | 0.05447 | 0.0545 | | 0.486 | | |
| | 0.05179 | 0.0518 | | 0.462 | | |
| 15 | 0.04774 | 0.0477 | 0.0503 ± 0.00271 | 0.426 | 0.449 ± 0.0242 | 33.8 |
| | 0.05011 | 0.0501 | | 0.447 | | |
| | 0.05314 | 0.0531 | | 0.474 | | |
| 30 | 0.04506 | 0.0451 | 0.0421 ± 0.00336 | 0.402 | 0.376 ± 0.0300 | 28.3 |
| | 0.03846 | 0.0385 | | 0.343 | | |
| | 0.04283 | 0.0428 | | 0.382 | | |

Human Donor 3

| | | | | | | |
|---|---|---|---|---|---|---|
| 0 | 0.51006 | 0.510 | 0.511 ± 0.00937 | 4.55 | 4.57 ± 0.0836 | 100 |
| (VC) | 0.50169 | 0.502 | | 4.48 | | |
| | 0.50986 | 0.510 | | 4.55 | | |
| | 0.52424 | 0.524 | | 4.68 | | |
| 5 | 0.31395 | 0.314 | 0.293 ± 0.0205 | 2.80 | 2.61 ± 0.183 | 57.2 |
| | 0.29125 | 0.291 | | 2.60 | | |
| | 0.27309 | 0.273 | | 2.44 | | |
| 15 | 0.28177 | 0.282 | 0.269 ± 0.0111 | 2.52 | 2.40 ± 0.0994 | 52.6 |
| | 0.26362 | 0.264 | | 2.35 | | |
| | 0.26154 | 0.262 | | 2.34 | | |
| 30 | 0.23625 | 0.236 | 0.229 ± 0.00670 | 2.11 | 2.04 ± 0.0598 | 44.7 |
| | 0.22559 | 0.226 | | 2.01 | | |
| | 0.22389 | 0.224 | | 2.00 | | |

Abbreviations:
SD, standard deviation;
VC, vehicle control (2% Water/1% Acetonitrile).
^{a}The observed analyzed value (μM) was below the lowest concentration on the standard curve (0.01 μM); values for Donor 1 based on the raw concentrations are included in parentheses in the mean concentration and percent of VC columns.
Note:
For all calculations above, the resulting values are shown with at least three significant figures for display purposes only.

TABLE 28
CYP2E1 Activity in Cryopreserved Human Hepatocyte Monolayers Following
48 hr Incubation with Quinine Sulfate Prior to Substrate Addition

| Quinine | 6-Hydroxychlorzoxazone formation | | | Specific Activity | | |
|---|---|---|---|---|---|---|
| Sulfate | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.22741 | 0.227 | 0.240 ± 0.0124 | 2.03 | 2.14 ± 0.110 | 100 |
| (VC) | 0.23443 | 0.234 | | 2.09 | | |
| | 0.25641 | 0.256 | | 2.29 | | |
| | 0.24004 | 0.240 | | 2.14 | | |
| 5 | 0.24596 | 0.246 | 0.249 ± 0.00280 | 2.20 | 2.22 ± 0.0250 | 104 |
| | 0.25076 | 0.251 | | 2.24 | | |
| | 0.25087 | 0.251 | | 2.24 | | |
| 15 | 0.28910 | 0.289 | 0.288 ± 0.00878 | 2.58 | 2.57 ± 0.0784 | 120 |
| | 0.29537 | 0.295 | | 2.64 | | |
| | 0.27803 | 0.278 | | 2.48 | | |
| 30 | 0.31180 | 0.312 | 0.349 ± 0.0322 | 2.78 | 3.12 ± 0.288 | 146 |
| | 0.36988 | 0.370 | | 3.30 | | |
| | 0.36505 | 0.365 | | 3.26 | | |

Human Donor 2

| | | | | | | |
|---|---|---|---|---|---|---|
| 0 | 0.09775 | 0.0978 | 0.0871 ± 0.00774 | 0.873 | 0.777 ± 0.0691 | 100 |
| (VC) | 0.08688 | 0.0869 | | 0.776 | | |
| | 0.08405 | 0.0841 | | 0.750 | | |
| | 0.07955 | 0.0796 | | 0.710 | | |
| 5 | 0.11735 | 0.117 | 0.118 ± 0.00125 | 1.05 | 1.06 ± 0.0112 | 136 |
| | 0.11756 | 0.118 | | 1.05 | | |
| | 0.11962 | 0.120 | | 1.07 | | |
| 15 | 0.15099 | 0.151 | 0.144 ± 0.00670 | 1.35 | 1.28 ± 0.0598 | 165 |
| | 0.14302 | 0.143 | | 1.28 | | |
| | 0.13768 | 0.138 | | 1.23 | | |
| 30 | 0.21984 | 0.220 | 0.212 ± 0.00776 | 1.96 | 1.89 ± 0.0693 | 243 |
| | 0.21059 | 0.211 | | 1.88 | | |
| | 0.20442 | 0.204 | | 1.83 | | |

Human Donor 3

| | | | | | | |
|---|---|---|---|---|---|---|
| 0 | 0.41024 | 0.410 | 0.397 ± 0.00989 | 3.66 | 3.55 ± 0.0883 | 100 |
| (VC) | 0.39244 | 0.392 | | 3.50 | | |
| | 0.38721 | 0.387 | | 3.46 | | |
| | 0.39834 | 0.398 | | 3.56 | | |
| 5 | 0.40711 | 0.407 | 0.473 ± 0.0570 | 3.63 | 4.22 ± 0.509 | 119 |
| | 0.51054 | 0.511 | | 4.56 | | |
| | 0.50051 | 0.501 | | 4.47 | | |
| 15 | 0.35252 | 0.353 | 0.358 ± 0.00770 | 3.15 | 3.20 ± 0.0688 | 90.1 |
| | 0.36670 | 0.367 | | 3.27 | | |
| | 0.35440 | 0.354 | | 3.16 | | |
| 30 | 0.40895 | 0.409 | 0.418 ± 0.0323 | 3.65 | 3.74 ± 0.289 | 105 |
| | 0.45442 | 0.454 | | 4.06 | | |
| | 0.39183 | 0.392 | | 3.50 | | |

Abbreviations:
SD, standard deviation;
VC, vehicle control (2% Water/1% Acetonitrile)
Note:
For all calculations above, the resulting values are shown with at least three significant figures for display purposes only.

TABLE 29
CYP3A4 Activity in Cryopreserved Human Hepatocyte Monolayers Following
48 hr Incubation with Quinine Sulfate Prior to Substrate Addition

| Quinine | 6β-Hydroxytestosterone formation | | | Specific Activity | | |
|---|---|---|---|---|---|---|
| Sulfate | Raw | Adjusted (μM) | | (pmol/min/million cells) | | Percent |
| (μM) | (μM) | Individual | Mean ± SD | Individual | Mean ± SD | of VC |

Human Donor 1

| | | | | | | |
|---|---|---|---|---|---|---|
| 0 | 0.03754^{a} | <0.100 | <0.100 ± 0.000 | <0.893 | <0.893 ± 0.000 | 100 |
| (VC) | 0.03861^{a} | <0.100 | (0.0367) | <0.893 | | |
| | 0.03223^{a} | <0.100 | | <0.893 | | |
| | 0.03851^{a} | <0.100 | | <0.893 | | |
| 5 | 0.04930^{a} | <0.100 | <0.100 ± 0.000 | <0.893 | <0.893 ± 0.000 | 100 |
| | 0.06117^{a} | <0.100 | (0.0597) | <0.893 | | (163) |
| | 0.06044^{a} | <0.100 | | <0.893 | | |
| 15 | 0.06639^{a} | <0.100 | <0.100 ± 0.000 | <0.893 | <0.893 ± 0.000 | 100 |
| | 0.07981^{a} | <0.100 | (0.0815) | <0.893 | | (222) |
| | 0.09425^{a} | <0.100 | | <0.893 | | |
| 30 | 0.06300^{a} | <0.100 | <0.100 ± 0.000 | <0.893 | <0.893 ± 0.000 | 100 |
| | 0.07508^{a} | <0.100 | (0.0741) | <0.893 | | (202) |
| | 0.08412^{a} | <0.100 | | <0.893 | | |

Human Donor 2

| | | | | | | |
|---|---|---|---|---|---|---|
| 0 | 0.37711 | 0.377 | 0.432 ± 0.0372 | 3.37 | 3.86 ± 0.332 | 100 |
| (VC) | 0.45023 | 0.450 | | 4.02 | | |
| | 0.44538 | 0.445 | | 3.98 | | |
| | 0.45707 | 0.457 | | 4.08 | | |
| 5 | 1.20397 | 1.20 | 1.40 ± 0.168 | 10.7 | 12.5 ± 1.50 | 323 |
| | 1.48926 | 1.49 | | 13.3 | | |
| | 1.50085 | 1.50 | | 13.4 | | |
| 15 | 1.94962 | 1.95 | 1.98 ± 0.0533 | 17.4 | 17.7 ± 0.476 | 459 |
| | 1.95787 | 1.96 | | 17.5 | | |
| | 2.04579 | 2.05 | | 18.3 | | |
| 30 | 1.34602 | 1.35 | 1.27 ± 0.0690 | 12.0 | 11.3 ± 0.616 | 293 |
| | 1.21990 | 1.22 | | 10.9 | | |
| | 1.23441 | 1.23 | | 11.0 | | |

Human Donor 3

| | | | | | | |
|---|---|---|---|---|---|---|
| 0 | 1.23104 | 1.23 | 1.22 ± 0.0378 | 11.0 | 10.9 ± 0.338 | 100 |
| (VC) | 1.18292 | 1.18 | | 10.6 | | |
| | 1.20885 | 1.21 | | 10.8 | | |
| | 1.27228 | 1.27 | | 11.4 | | |
| 5 | 4.37682 | 4.38 | 4.48 ± 0.150 | 39.1 | 40.0 ± 1.34 | 366 |
| | 4.40376 | 4.40 | | 39.3 | | |
| | 4.64918 | 4.65 | | 41.5 | | |
| 15 | 7.74794 | 7.75 | 7.68 ± 0.0583 | 69.2 | 68.6 ± 0.520 | 628 |
| | 7.64217 | 7.64 | | 68.2 | | |
| | 7.65262 | 7.65 | | 68.3 | | |
| 30 | 6.78923 | 6.79 | 6.65 ± 0.120 | 60.6 | 59.4 ± 1.07 | 544 |
| | 6.61287 | 6.61 | | 59.0 | | |
| | 6.56027 | 6.56 | | 58.6 | | |

Abbreviations:
SD, standard deviation;
VC, vehicle control (2% Water/1% Acetonitrile).
^{a}The observed analyzed value (μM) was below the lowest concentration on the standard curve (0.1 μM); values for Donor 1 based on the raw concentrations are included in parentheses in the mean concentration and percent of VC columns.
Note:
For all calculations above, the resulting values are shown with at least three significant figures for display purposes only.

Quinine sulfate at the tested concentrations induced CYP1A2 activity in human hepatocytes prepared from all three donors (Table 21), with increasing induction of CYP1A2 activity observed with increasing quinine sulfate concentration. The maximal induction observed for the 3 sets of hepatocytes ranged from 4- to 21-fold at 30 μM of quinine sulfate.

CYP2A6 activity in cryopreserved human hepatocytes was quantified by adding coumarin to the hepatocytes and measuring the formation of 7-hydroxycoumarin (7-HC), as well as each of the conjugated derivatives of 7-HC: 7-hydroxycoumarin glucuronide (7-HCG) and 7-hydroxycoumarin sulfate (7-HCS). In hepatocytes from Donor 1 under these experimental conditions, there was no detectable amount of 7-HC and 7-HCS in hepatocytes in the vehicle control or treated with quinine sulfate (Tables 22a & 22c). However, quinine sulfate increased the formation of 7-HCG in hepatocytes from Donor 1 (Table 22b). Quinine sulfate increased the formation of 7-HCG and 7-HCS in hepatocytes from Donor 2 (Tables 22b & 22c). Based on the total measured concentrations of metabolites formed in the hepatocytes, quinine sulfate at the tested concentrations induced CYP2A6 activity in hepatocytes prepared from Donor 2 (Table 22d), however this observation is primarily a result of the induction effects on formation of 7-HCG and 7-HCS. Measured levels of 7-HC (Table 22a), however, were below the lowest concentration standard for the vehicle control and test samples and therefore did not show statistically significant induction. Quinine sulfate at 5 μM induced CYP2A6 activity as measured by total measured concentrations of metabolites formed in hepatocytes prepared from Donor 3; this was due primarily to the induction effects of quinine sulfate at that concentration on the formation of 7-HCG (Table 22b), although 7-HC also showed a similar % induction (Table 22a), but it was not statistically significant (p>0.05). All three metabolites (7-HC, 7-HCG, and 7-HCS), as well as the total, showed decreasing levels of metabolite formed with increasing quinine sulfate. At 30 μM quinine sulfate, a significant level of inhibition of CYP2A6 activity in hepatocytes from Donor 3 was observed, as measured by each metabolite individually or in composite. The quinine sulfate induction in the two donors was less than 1-fold.

CYP2B6 activity in cryopreserved human hepatocytes was quantified by adding 1 mM S-mephenytoin to the hepatocytes and measuring the formation of the CYP2B6-specific metabolite, nirvanol. Quinine sulfate at the tested concentrations did not induce CYP2B6 activity in human hepatocytes prepared from Donor 1 (Table 23). Quinine sulfate produced increasing induction of CYP2B6 activity in hepatocytes prepared from Donor 2 with increasing concentration at 5 and 15 μM (Table 23), however the CYP2B6 activity at 30 μM quinine sulfate did not differ from the vehicle control at a statistically significant level (p>0.05). Quinine sulfate induced CYP2B6 activity in hepatocytes prepared from Donor 3 at a statistically significant level at the tested concentrations (Table 23). Quinine sulfate induced activities of CYP2B6 in two of the three donors tested, however the induction was less than 1-fold.

Quinine sulfate at the tested concentrations did not induce CYP2C8 activity in human hepatocytes isolated from Donor 1 (Table 24). The apparent increase of CYP2C8 activity in Donor 1 following treatment with 30 μM quinine sulfate was not statistically significant (p=0.052; unpaired two-tailed t test). Quinine sulfate at 5 μM induced CYP2C8 activity from hepatocytes prepared from Donors 2 and 3 at a statistically significant level. At the two higher concentrations, CYP2C8 activity from hepatocytes prepared from Donor 2 showed apparent inhibition, but it was not statistically significant (p>0.05, unpaired two-tailed t test). At 15 μM, the apparent induction of CYP2C8 activity from hepatocytes prepared from Donor 3 was not statistically significant (p>0.05, unpaired two-tailed t test), while 30 μM quinine sulfate produced statistically significant inhibition of CYP2C8 activity from hepatocytes prepared from Donor 3 (Table 24).

Quinine sulfate at 5 μM did not increase CYP2C9 activity (Table 25) in human hepatocytes isolated from Donor 1 at a statistically significant level (p>0.05; unpaired two-tailed t test). However, induction of CYP2C9 activity occurred in the Donor 1 hepatocytes at the increased concentrations of quinine sulfate. Quinine sulfate at all tested concentrations produced statistically significant induction of CYP2C9 activity from hepatocytes prepared from Donors 2 and 3 (Table 25).

Quinine sulfate at each of the tested concentrations induced CYP2C19 activity in hepatocytes prepared from Donor 3 at a statistically significant level (Table 26). CYP2C19 activity levels in hepatocytes isolated from Donors 1 and 2 were undetectable in the vehicle controls and for each tested concentration of quinine sulfate (Table 26). As noted above, the reference control with 25 μM rifampin for each of these two donors also did not show significant induction.

Quinine sulfate induced activities of CYP2C9 (all three donors) and CYP2C19 (one of the three donors), and also CYP2C8 at one concentration (two of the three donors). However, the observed induction of each of these three enzymes was less than 0.5-fold.

Formation of the metabolite dextrorphan by CYP2D6 activity in the hepatocytes from Donor 1 for the vehicle control and at each quinine sulfate concentration tested was measurable, but below the concentration of the lowest standard for the standard curve (Table 27). Using these measured values, each concentration of quinine sulfated inhibited CYP2D6 activity at a statistically significant level, with the percent of the vehicle control being 76.3, 88.2, and 78.5% at 5, 15, and 30 μM quinine sulfate, respectively. Quinine sulfate at the concentrations tested clearly inhibited CYP2D6 activity in human hepatocytes isolated from Donors 2 and 3 (Table 27). Quinine sulfate inhibited CYP2D6 activity when pre-incubated with the enzymes, prior to addition of the isozyme-specific substrate, or when added roughly simultaneously with the isozyme-specific substrate, as seen in Example 2 above.

At least one tested quinine sulfate concentration induced CYP2E1 activity in all three donors (Table 28), however the maximal induction was about 1.5-fold. Although quinine sulfate at 5 μM did not induce CYP2E1 activity in human hepatocytes isolated from Donor 1 at a statistically significant level (p>0.05 in an unpaired t-test), statistically significant induction occurred as the concentration of quinine sulfate increased (Table 28). For hepatocytes prepared from Donor 2, quinine sulfate produced increasing induction of the CYP2E1 activity with increasing concentration (Table 28). Observed induction of CYP2E1 activity in hepatocytes prepared from Donor 3 was significant only at 5 μM quinine sulfate; the small apparent increase in metabolite formed at 30 μM quinine sulfate is not statistically significant (p>0.05 in an unpaired t-test). The apparent inhibition at 15 μM quinine sulfate is statistically significant (Table 28).

Formation of the metabolite 6β-Hydroxytestosterone by CYP3A4 activity in the hepatocytes from Donor 1 for the vehicle control and at each quinine sulfate concentration tested was measurable, but below the concentration of the lowest standard for the standard curve (Table 29). Using these measured values, each concentration of quinine sulfated induced CYP23A4 activity at a statistically significant level, with the percent of the vehicle control being 163, 222, and 202% at 5, 15, and 30 μM quinine sulfate, respectively. Quinine sulfate at the tested concentrations also induced CYP3A4 activity in human hepatocytes prepared from Donors 2 and 3 at statistically significant levels. The maximal induction was about 5-fold.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of optimizing safe use of quinine, comprising
informing a patient or the patient's medical care worker that
a) quinine is metabolized by cytochrome p450 1A2;
b) quinine is an inhibitor of cytochrome p450 1A2, 2B6, or 2C9;
c) quinine is an inducer of CYP2A6, CYP2B6, CYP2C9, or CYP2E1;
d) quinine is not an inhibitor of CYP2E1;
e) quinine is not an inducer of CYP2D6 or CYP2C19; or
f) quinine affects activity of CYP2B6, CYP2C9, or CYP2E1, and
administering quinine to the patient such that a side effect, an adverse event, or an active agent interaction is minimized,
wherein the patient is a patient with uncomplicated P. falciparum malaria, malaria caused by Plasmodium species, severe or complicated Plasmodium falciparum malaria, leg cramps, or babesiosis.

2. The method of claim 1, wherein the informing is by
providing published material;
providing a product insert, a flyer, or an advertisement;
a seminar, conference presentation, or other educational presentation; or
a conversation between a pharmaceutical sales representative and a medical care worker.

3.-5. (canceled)

6. The method of claim 1 wherein the patient or the patient's medical care worker is a human patient.

7. (canceled)

8. The method of claim 1, wherein the patient is receiving quinine therapy.

9. The method of claim 1, additionally comprising informing the patient or the patient's medical care worker that
administration of quinine with a substance that is an inhibitor of CYP1A2 can result in increased plasma concentration of quinine;
administration of quinine with a substance that is an inducer of CYP1A2 can result in decreased plasma concentration of quinine;
administration of quinine with a substance that is an inhibitor or an inducer of CYP1A2 or that affects the activity of CYP2A6, CYP2B6, CYP2C9, or CYP2E1 can affect plasma concentration, bioavailability, safety, efficacy, or a combination comprising at least one of the foregoing of quinine or the substance;
administration of quinine with a substance that is a substrate of CYP2D6 or CYP2C19 is unlikely to result in reduced plasma concentration of the substance;
administration of quinine with a substrate of CYP2E1 is unlikely to result in increased plasma concentration of the substance;
administration of quinine with a substance that is a substrate of CYP2A6, CYP2B6, CYP2C9, or CYP2E1 can result in decreased plasma concentration of the substance; or
administration of quinine with a substance that is a substrate of CYP1A2, CYP2B6, or CYP2C9 can result in increased plasma concentration of the substance.

10. The method of claim 9, wherein the substance is an active agent.

11. The method of claim 9, wherein the substance is a substrate of CYP1A2, CYP2A6, CYP2B6, CYP2C9, or CYP2E1.

12. The method of claim 9, wherein the substance is aminophylline, cyclophosphamide, cyclosporine, efavirenz, fosphenytoin, glimepiride, mexiletine, phenytoin, progesterone, tamoxifen, theophylline, thioridazine, or warfarin.

13. The method of claim 1, wherein the method further comprises
determining the metabolizer phenotype of the patient for CYP2A6, CYP2B6, or CYP2C9.

14.-46. (canceled)

47. The method of claim 1, additionally comprising
administering an active agent that is an inhibitor or an inducer of CYP1A2 or that affects activity of CYP2A6, CYP2B6, CYP2C9, or CYP2E1 to the patient; and
monitoring the patient's plasma concentration of the active agent or quinine.

48.-60. (canceled)

61. The method of claim 47, additionally comprising
altering dosing of quinine or the active agent based on the determined plasma concentration of quinine or the active agent.

62. The method of claim 1, wherein a substance that is an inhibitor or an inducer of CYP1A2 or that affects activity of CYP2A6, CYP2B6, CYP2C9, or CYP2E1 is administered to the patient with quinine.
